(19) United States
(12) Patent Application Publication
(10) Pub. No.: US 2019/0091198 A1
(43) Pub. Date: Mar. 28, 2019

(54) CANNABIONID AND CANNABIS-BASED COMPOSITIONS AND METHODS FOR THE TREATMENT OF INFLAMMATORY CONDITIONS OF THE GASTROINTESTINAL TRACT

(71) Applicants: Lihi BAR-LEV SCHLEIDER, Moshav Yevul (IL); TO PHARMACEUTICALS LLC, Wilmington, DE (US)

(72) Inventor: Lihi BAR-LEV SCHLEIDER, Moshav Yevul (IL)

(21) Appl. No.: 16/086,338

(22) PCT Filed: Mar. 28, 2017
(86) PCT No.: PCT/IL2017/050388

§ 371 Date: Sep. 19, 2018

Related U.S. Application Data
(60) Provisional application No. 62/313,882, filed on Mar. 28, 2016.

(51) Int. Cl. A61K 31/352; A61K 31/05; A61K 45/06; A61K 36/185; A61P 1/04

(52) U.S. Cl. CPC A61K 31/352; A61K 31/05; A61P 1/04; A61K 36/185; A61K 45/06

(57) ABSTRACT

The invention provides compositions and methods for treating inflammatory condition of the gastrointestinal (GI) tract, specifically those related to Inflammatory Bowel Disease (IDB). Compositions according to the invention due to their specific content of cannabinoids and methods comprising specific modes of administration thereof are particularly applicable to the treatment of the two major IDBs, Crohn's disease and colitis.

### TECHNOLOGICAL FIELD

The invention pertains to pharmaceutical compositions comprising cannabinoids and Cannabis-based formulations, and further to methods using thereof for the treatment of inflammatory conditions of the gastrointestinal (GI) tract, specifically Inflammatory Bowel Disease (IBD), including Crohn's Disease and Ulcerative Colitis.

### BACKGROUND

Medicinal value of Cannabis is well documented in literature. Cannabinoids, the active ingredients of Cannabis, are found in the resin-producing pistillate inflorescences of female Cannabis plants. Various types of Cannabis, such as Cannabis Sativa and Cannabis Indica, may contain 60 to 80 different kinds of cannabinoids, notable examples of which are tetrahydrocannabinol (THC) and cannabidiol (CBD). These two cannabinoids have been related to many distinct pharmacological effects, including analgesic, antiemetic, antioxidative, neuroprotective, and anti-inflammatory activities in various normal and abnormal cells and tissues. The discovery of the endogenous cannabinoid system with specific receptors and ligands in the brain, and in peripheral tissues has led to understanding that the endocannabinoid system represents a previously unrecognized ubiquitous homeostatic network. At least two molecular receptor proteins (CB1 and CB2) and two endogenous cannabinoids (anandamide and 2-acylglycerol) have been identified in numerous body tissues, including the neural and immune systems. It now appears that the endocannabinoid system evolved with our species and is intricately involved in normal human physiology, specifically in movement control, pain, appetite, memory, immunity and inflammation, among others. This explains the tremendous potential of exogenous cannabinoids and Cannabis-based medicines for the treatment of various human disorders.

A number of oral formulations of cannabinoids are commercially available today by prescription for specific clinical indications. Marinol capsules containing dronabinol, a synthetic Δ⁹-THC, in sesame oil were approved in various countries for use as an antiemetic in patients subjected to cancer chemotherapy, and for appetite stimulation in AIDS patients suffering from wasting syndrome. Cesamet capsules comprising nabilone, a synthetic THC analog, were recently approved as a Marinol substitute. Namisol tablets containing pure THC, Arvisol tablets containing CBD and Sativex (nabiximols) an oral spray containing THC and CBD, are more recent Cannabis-based formulations approved for a number of indications including Alzheimer's disease, chronic neural pain, and multiple sclerosis.

The instant invention applies to a group of inflammatory conditions collectively referred to as Inflammatory Bowel Disease (IBD), affecting the gastrointestinal (GI) tract and emerging, most likely, due to an autoimmunity. The two major types of IBD are ulcerative colitis and Crohn's disease. Ulcerative colitis involves predominantly the colon or large intestine, and Crohn's disease—any part of the GI tract, most commonly the small intestine or colon, or both. The main symptoms of ulcerative colitis and Crohn's disease are similar, including pain, swelling or cramping, recurring or bloody diarrhea, weight loss and extreme tiredness. IBD is usually diagnosed in people in their late teens or early 20s, but can affect people of any age. According to CDC, there are about 1-1.3 million people suffering from IBD, in the US alone. The prevalence of Crohn's disease has been estimated at 200 per 100,000 adults, and ulcerative colitis—at 230 per 100,000 adults.

There is currently no definite cure for IBD, the majority of treatments constitute a palliative care. Traditional pharmacological treatments for IBD include, aminosalicylates or corticosteroids to reduce inflammation, and immunosuppressants to reduce activity of the immune system. About 20% of patients with severe symptoms of ulcerative colitis are non-responders and are usually referred to a surgical removal of the inflamed section. In Crohn's disease—about 60-75% of patients may be referred to a surgery.

The inventors have previously noted that certain extracts of Cannabis can be effective for IBD, and for Crohn's disease in particular [1, 2], and that certain strains may have anti-inflammatory and nociceptive effects in an animal model of inflammation of non-GI origin [3]. Notwithstanding, these and other human studies using Cannabis were relying to great extent on trial and error, leaving dosing and modes of administration highly individualized.

A Cannabis administered via smoking has the advantage of rapid onset of effect and easy dose titration. However, the guidelines for precise dosing of smoked or vaporized Cannabis have not been yet established. Cannabis may be consumed in baked goods, such as cookies, or drunk as teas or infusions. The absorption of these products, in contrast, is slow and erratic, and the onset of effects lasting much longer compared to smoking. For other dosage forms, e.g., butters, oils, creams and ointments, similarly, no dosing information is currently available and much of the information is anecdotal in nature. Dosing schemes developed on the basis of the known chemistry and pharmacology of Cannabis still suffer from considerable controversy.

One prevailing notion, however, is that route of administration is an important determinant of pharmacokinetics of various cannabinoids in Cannabis, particularly the absorption and metabolism.

Despite the general belief that Cannabis is safe and no lethal doses of Cannabis have been reported so far, Cannabis is still considered a hazardous drug. Adverse effects of Cannabis include: cognitive and memory impairments, changes in mood, altered perception, decreased impulse control, particularly during adolescence, occupational hazards, fluctuations of blood pressure, syncope or tachycardia, respiratory insufficiency particularly with smoked Cannabis, increased severity of steatosis in patients with liver or renal disease, pregnancy complications in women, and borderline of infertility in men. There are also significant drug-drug interactions, most notable with sedative-hypnotics and alcohol. Patients with no prior experience with Cannabis are usually cautioned to begin at a very low dose and to stop therapy if unacceptable or undesirable side effects occur. Thus the management of risk/benefits of medicinal Cannabis, and also of the commercial purified oral formulations, is still difficult.

Thus, there is an urgent need for standards to be set that would maximize benefits and minimize risks of using medicinal Cannabis for specific clinical indications, which is all the more critical in view of complexity of cannabinoid pharmacology, inter-individual differences in cannabinoid receptors distribution, density and function, cannabinoid metabolism and bioavailability, and heterogeneity of cannabinoid content in various Cannabis plants. The instant invention is meant to address these issues, specifically in the context of IBD.

### REFERENCES

1. Naftali T et al., ‘Treatment of Crohn's Disease with Cannabis: An Observational Study’, Israeli Medical Association Journal (IMAJ) 2011; 13:455-458, described a retrospective study based on self-reporting of 20 patients suffering from Crohn's disease that were granted a license for medical Cannabis treatment.
2. Naftali T et al., ‘Cannabis Induces a Clinical Response in Patients With Crohn's Disease: A Prospective Placebo-Controlled Study’, Clinical Gastroenterology and Hepatology 2013; 11:1276-1280, described study of 21 patients granted a license for medical cannabis for the treatment of Crohn's disease. The primary end point of the study was not achieved.
3. Gallily R et al., ‘Overcoming the Bell Sjaped Dose-Response of Cannobidiol by using Cannabis Extract enriched in Cannabidiol’, Pharmacology & Pharmacy 2015; 6:75-85, described a specific strain of Cannabis particularly enriched in CBD as being capable of certain anti-inflammatory and nociceptive effects in animal models.

### GENERAL DESCRIPTION

The instant invention stems from accumulated experience of the inventors with cultivation of novel strains of medical Cannabis and development of controlled methods using thereof for specific clinical indications. In connection with certain embodiments the invention relates to three Cannabis cultivars, or three distinct groups of Cannabis plants, certain members of which were generally described in terms of morphological features and cultivation conditions (see below). These cultivars, including additional members, are now described in relation to surprising clinical properties and clinical uses in the context of IBD. Members of these three cultivars are referred to throughout this document by trade names. The referenced US Plant patent applications are herein incorporated by reference, including the applications derived therefrom, i.e., continuation or continuation in part applications.

Thus in certain aspects the invention relates to:

i. A phyto-derived material obtained from a Cannabis strain enriched in THC in an amount ranging from 16 and 24% per weight (w/w), with relatively low CBD, particularly in the resin-producing flowers of female plants. An exemplary member of this group referred to herein as Erez was generally described in the US Plant Patent Application No. 2014/0245494.

ii. A phyto-derived material obtained from a Cannabis strain enriched in CBD and particularly low THC, in amounts ranging from 15 and 16.5% and 0.8 and 3.75% (w/w), respectively. An exemplary member of this group referred to herein as Avidekel was generally described in the US Plant Patent Application No. 2014/0259228.

iii. A phyto-derived material obtained from a Cannabis strain having substantially equal ratio of THC:CBD in an amount ranging from 10 and 13% and 8 and 12.5% (w/w), respectively. An exemplary member of this group referred to herein as Midnight was generally described in the US Plant Patent Application No. 2014/0245495.

It has been presently demonstrated that preparations produced from exemplary members of the above cultivars have specific therapeutic effects in patients with IBD, i.e., Crohn's disease and colitis, revealed by significant improvement of Disease Activity Index (DAI) scores, indices of inflammation according to blood and intestinal disease specific markers, accompanied by a reduction of adverse effects and improvement in general quality of life.

Most notably according to the invention the above therapeutic effects could be enhanced and prolonged by applying certain dosing of administration regimens in the form of mono and combination therapies.

Present studies have shown that a phyto-derived material enriched in CBD, for example an oil-based extract of a material derived from Avidekel or other members of this group, is surprisingly effective for prolonged treatment and management of IBD. Such CBD enriched material is particularly effective for the treatment of Crohn's disease. Compositions based on Avidekel and other members of this group are particularly interesting due to scarcity of psychotropic effects and their ensuing suitability to day dosing.

The studies have further demonstrated that a phyto-derived material enriched in THC, for example a material derived from Erez or other members of this group in the form of cigarettes, is also effective for IBD, particularly immediate alleviation of one or more IBD symptoms. Such THC enriched material was particularly beneficial for patients with ulcerative colitis. Erez-based compositions and alike, because of their high THC content, are particularly suitable for night dosing.

An alternative treatment revealed in present studies involved a phyto-derived material containing THC and CBD in equal or substantially equal quantities, exemplified by a material derived from Midnight in the form of cigarettes, for example. This kind of preparation proved to be an effective substitute for THC enriched material (such as Erez), particularly in patients uncompliant with psychotropic effects of THC. Due to their THC:CBD content, Midnight-based compositions and alike are suitable for both, day and night dosing.

Most notably, the presently described study of about 300 patients with IBD, half of which treated for more than 6 months, has demonstrated that in the majority of patients a combination of preparations derived from Avidekel, Erez and/or Midnight proved to be the most effective for immediate as well as prolonged alleviation and treatment, and long term management of IBD.

These findings apply on several levels:

First, in terms of specific cannabinoid compositions comprising certain proportions of THC and CBD to be applied for the treatment of IBD and IBD related conditions;

Second, in terms of specific Cannabis-based compositions used in specific formulations, doses and administration routes to provide immediate relief and long-term management of IBD;

Third, in terms of specific cannabinoid compositions to be applied as specific treatments for Crohn's disease or colitis.

This latter point is particularly surprising in view of difficulties with differential diagnosis of these disorders.

The above have laid the basis for the following disclosure of the invention. It should be noted that any of the embodiments and aspects described herein can be used in conjunction with one another, unless otherwise indicated or apparent from the context. Other embodiments will become apparent to those skilled in the art from a review of the ensuing description.

### BRIEF DESCRIPTION OF FIGURES

FIGS. 1A-1I illustrate specific embodiments of the invention in connection with the treatment of Crohn's disease. Figures describe clinical outcomes of a prospective study of patients with active Crohn's disease, including patients treated with Avidekel-derived oil extract comprising THC 4% and CBD 16% (ratio 1:4) (N=18) or a placebo (N=21), administered orally. Figures display general trends observed during 8 weeks follow up and 2 week wash out period in the treatment (solid black lines) vs. the placebo (dotted lines) groups. FIG. 1A relates to assessment of Crohn's Disease Activity Index (CDAI); FIG. 1B relates to mental health status and reporting on side effects according to Quality of Life (SF-36) questionnaires; FIG. 1C-1F relate to general clinical parameters, i.e., patients' weight, levels of White Blood Cells (WBC), Hemoglobin (HB) and hematocrit (HCT); FIG. 1G-1I relate to clinical parameters specific to Crohn's disease, i.e., levels of C-reactive protein (CRP) in the blood (a marker of inflammation); fecal Calprotectin (a marker of intestinal inflammation); and SES colonoscopy scores.

FIGS. 2A-2D illustrate further embodiments of the invention in connection with pharmacokinetic profiles of Avidekel-derived oil extract, including two main cannabinoids, THC (Δ⁹-THC) and CBD, and two metabolites, 11-Hydroxy Δ⁹-THC (active metabolite) and Δ⁹ Carboxy THC (inactive metabolite). Pharmacokinetic studies were performed in a sub-group of patients from the study described in FIGS. 1A-1I. Specifically: FIG. 2A shows mean blood (serum) levels of THC (Δ⁹-THC) (ng/mL) in time points 15, 30, 45, 60 and 90 min, and 2, 3, 4, 5 and 6 h. measured by LC-MS/MS (N=7); FIG. 2C-2D shows analogous profiles relating to CBD (ng/mL), 11-Hydroxy Δ⁹-THC and Δ⁹ Carboxy THC, respectively, in the same group.

FIGS. 3A-3I illustrates a further embodiments of the invention in connection with the treatment of colitis. Figures describe clinical outcomes of patients with ulcerative colitis treated with Erez-derived material enriched in THC (23%) (N=14) or placebo cigarettes (N=13), administered by smoking. Figures display general trends observed in the treated (solid black lines) and the placebo (dotted lines) groups after analogous FIGS. 1A-1I.

### DETAILED DESCRIPTION OF EMBODIMENTS

Before describing the invention it should be noted that it is not limited to herein described methods and experimental conditions, as well as the terminology used herein for describing particular embodiments is not intended to be limiting. Unless defined otherwise, all technical and scientific terms used herein have the meaning as commonly understood by one of ordinary skill in the art to which this invention pertains. Although any methods and materials similar or equivalent to those described herein can be used in the practice or testing of the invention, particular methods and materials are now described.

The instant invention generally relates to compositions comprising certain ratios of THC:CBD for use in a method for treating, alleviating or reducing IBD and IBD related conditions, or at least one symptom related to IBD. Such compositions may further comprise carriers, buffers, excipients.

In other words, the invention pertains to a group of intestinal disorders communally referred to as ‘Inflammatory Bowel Disease’ (IBD) or condition, characterized with a prolonged regional enteritis of the GI tract, including the mouth, esophagus, stomach, small intestine and/or large intestine. Notable members of this group are Crohn's disease and ulcerative colitis, and related conditions such as Irritable Bowel Syndrome (IBS). Differential diagnosis of patients that can benefit from the presently described compositions and methods is made by a treating physician on the basis of physical examination, anamnesis and one or more diagnostic tests, including stool and blood tests, a biopsy, and medical imaging using X ray, flexible sigmoidoscopy, colonoscopy, capsule endoscopy, CT or MRI.

Crohn's disease is usually noncontiguous having skipped areas of a normal mucosa. The ulcerations in Crohn disease tend to be linear and often lead to the classic cobblestone appearance of the mucosa. Granulomas are present in 60% of Crohn's disease and almost never present in ulcerative colitis. The inflammation in Crohn's can be transmural, whereas in ulcerative colitis it is confined to the mucosa and submucosa. Crohn's disease may involve the entire GI tract, whereas ulcerative colitis involves only the large bowel. Approximately 90% of patients with Crohn's disease have involvement of the terminal ileum and/or right colon. Pediatric patients are more likely to present with disease limited to the small intestine, although very young children often present with purely colonic disease. A variety of intestinal and extraintestinal manifestations may be observed in conjunction with either Crohn's disease or colitis. Features differentiating these two forms of IBDs are summarized in Table 1.

TABLE 1
Distinguishing Ulcerative Colitis from Crohn Disease

| Ulcerative Colitis | Crohn Disease |
|---|---|
| Only colon involved | Panintestinal |
| Continuous inflammation extending | Skip-lesions with intervening |
| proximally | normal |
| Inflammation in mucosa and | Transmural inflammation |
| submucosa only | |
| No granulomas | Noncaseating granulomas |
| Perinuclear ANCA (pANCA) positive | ASCA positive |
| Bleeding (common) | Bleeding (uncommon) |
| Fistulae (rare) | Fistulae (common) |
| Weight loss (common) | Weight loss (uncommon) |
| Obstruction (common) | Obstruction (common) |

In certain embodiments, compositions and methods of the invention can apply to other types of colitis, such as idiopathic colitis (e.g. lymphocytic colitis, collagenous colitis, chemical colitis), ischemic colitis and infectious colitis (e.g. Clostridium difficile, Shigella dysenteriae), and undeterminable type or atypical colitis.

In yet other embodiments, the invention can apply to IBD related disorders. One of common conditions of this group is Irritable Bowel Syndrome (IBS), a spectrum of disorders characterized by the presence of chronic abdominal pain and/or discomfort and alterations in bowel habits, including diarrhea predominant (D-IBS), constipation predominant (C-IBS), and a mixed pattern (M-IBS) types.

In its the broadest sense the invention provides cannabinoid compositions that are applicable to the treatment of IBD using therapeutic methods of the invention. The term ‘cannabinoids’ encompasses herein endocannabinoids, phytocannabinoids or synthetic cannabinoids. Specific cannabinoids include, e.g., THC, CBD and others, as well as encompasses synthetic, semi-synthetic and natural cannabinoid (i.e. purified or extracted from a Cannabis plant).

In its main aspects, the invention pertains to tetrahydrocannabinol-type (THC), cannabidiol-type (CBD) and cannabinol-type (CBN) cannabinoids.

‘Tetrahydrocannabinol’ (THC) refers herein to a class of psychoactive cannabinoids characterized by high affinity to CB1 and CB2 receptors, a molecular formula C₂₁H₃₀O₂, an average mass of approximately 314.46 Da and a general structure of Formula I.

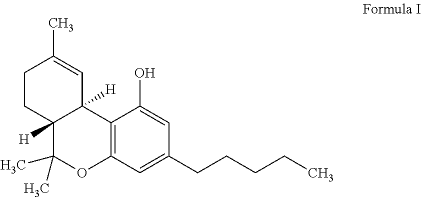 [SMILES: CCCCCc1cc(O)c2c(c1)OC(C)(C)[C@@H]1CCC(C)=C[C@@H]21]

‘Cannabidiol’ (CBD) refers herein to a class of non-psychoactive cannabinoids with low affinity to CB1 and CB2 receptors, a formula C₂₁H₃₀O₂, an average mass of 314.46 Da and a general structure of Formula II.

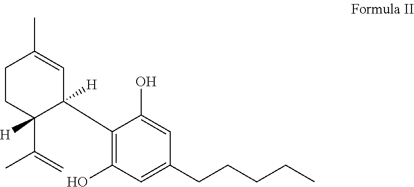 [SMILES: C=C(C)[C@@H]1CCC(C)=C[C@H]1c1c(O)cc(CCCCC)cc1O]

‘Cannabinol’ (CBN) refers to a class of weak psychoactive cannabinoids acting as partial agonists of THC at the CB1 receptors and CB2 receptors, with a formula C₂₁H₂₆O₂, an average mass 310.19 Da and a general structure of Formula III.

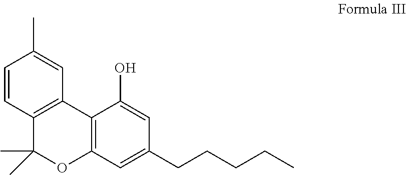 [SMILES: CCCCCc1cc(O)c2c(c1)OC(C)(C)c1ccc(C)cc1-2]

The terms ‘THC’, ‘CBD’, ‘CBN’ herein encompass isomers, derivatives or precursors, such as (−)-trans-Δ9-tetrahydrocannabinol (Δ9-THC), Δ8-THC, and Δ9-CBD, and also to THC and CBD derived from their respective 2-carboxylic acids (2-COOH), THC-A and CBD-A.

In its numerous compositions, the invention provides compositions derived from or based on the use of a cannabis plant, and thus may be regarded as ‘phyto-derived compositions’ or phyto-derived materials. This term encompasses herbal preparations, concentrated extracts and purified products. Regarding extracts, there are number of methods for producing a concentrated cannabis-derived material, e.g., a filtration, an ice water extraction, butane extraction or CO₂ extraction processes, oil extracts made by a solvent evaporation. One of the main sources of cannabinoids is a resin-producing pistillate inflorescences of a female Cannabis plant.

It should be noted in this connection that the presently exemplified preparations of Avidekel in oil were essentially distinct from the original plants by the proportion of active ingredients, e.g., THC and CBD (see Tables 3 and 4).

It should be further noted in this connection that a phyto-derived material and extracts thereof comprise apart from the presently identified active ingredients THC, CBD and CBN, additional cannabinoids and other constituents of plant origin (e.g., terpenes), contributing to distinctive properties thereof in therapeutic impact and applications (see Table 3 and EXAMPLES 2 and 3).

Thus, in its many different aspects the invention provides a phyto-derived composition comprising at least one cannabinoid and at least one terpene for use in a method of treating, alleviating or reducing at least one symptom of IBD, wherein

(a) said composition is derived from at least one of a cannabis plant enriched in THC, a cannabis plant enriched in CBD, a cannabis plant wherein the amounts of THC and CBD are substantially equal,

(b) wherein at least one cannabinoid is selected from THC, CBD, and CBN, and

(c) wherein said at least one terpene is selected from monoterpenes and sesquiterpenes.

In some embodiments, compositions of the invention are derived from a female cannabis plant in a dosage form of an oil extract or a dry material, both of which have been presently exemplified.

As has been noted, compositions of the invention can comprise additional cannabinoids of plant origin. The main classes of natural cannabinoids are listed in Table 2 below.

In other words, in numerous embodiments compositions of the invention can comprise a tetrahydrocannabinol-type and cannabinol-type (THC, CBN), a cannabidiol-type (CBD), a cannabigerol-type (CBG), a cannabichromene-type (CBC), a cannabielsoin-type (CBE), an iso-tetrahydrocannabinol-type (iso-THC), a cannabicyclol-type (CBL), a cannabicitran-type (CBT), a derivative, a precursor, or a combination thereof.

TABLE 2
Main classes of natural cannabinoids

| Type | Skeleton |
|---|---|
| Cannabigerol-type CBG | 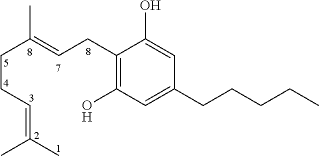 [SMILES: CCCCCc1cc(O)c(C/C=C(\C)CCC=C(C)C)c(O)c1] |
| Cannabichromene-type CBC | 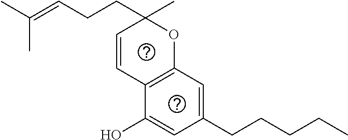 [SMILES: CCCCCc1cc(O)c2c(c1)OC(C)(CCC=C(C)C)C=C2] |
| Cannabidiol type CBD | 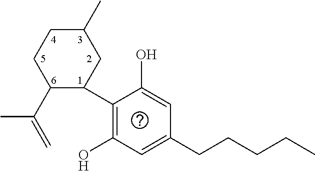 [SMILES: C=C(C)C1CCC(C)CC1c1c(O)cc(CCCCC)cc1O] |
| Tetrahydrocannabinol-and Cannabinol-type THC, CBN | 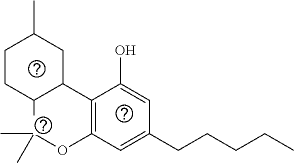 [SMILES: CCCCCc1cc(O)c2c(c1)OC(C)(C)C1CCC(C)CC21] |
| Cannabielsoin-type CBE | 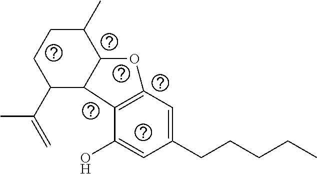 [SMILES: C=C(C)C1CCC(C)C2Oc3cc(CCCCC)cc(O)c3C12] |
| iso-Tetrahydrocannabinol-type iso-THC | 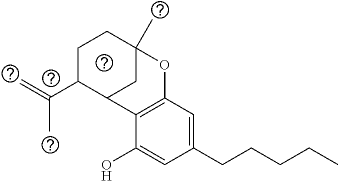 [SMILES: C=C(C)C1CCC2(C)CC1c1c(O)cc(CCCCC)cc1O2] |
| Cannabicyclol-type CBL | 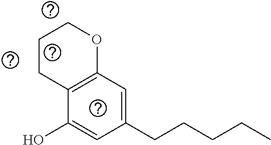 [SMILES: CCCCCc1cc(O)c2c(c1)OCCC2] |
| Cannabicitran-type CBT | 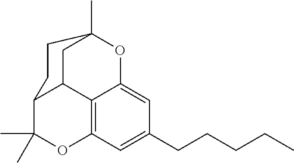 [SMILES: CCCCCc1cc2c3c(c1)OC(C)(C)C1CCC(C)(CC31)O2] |

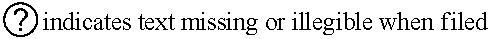

All classes derive from a cannabigerol-type compound and differ mainly how the precursor is cyclized. The classical cannabinoids are derived from their respective 2-carboxylic acids (2-COOH, also denoted with -A) by decarboxylation (catalyzed by heat, light, or alkaline conditions). Tetrahydrocannabinol and cannabidiol acid precursors, THC-A and CBD-A are also relevant to the invention. A number of relevant phytocannabinoids are listed below:
THC (Tetrahydrocannabinol, including the two isoforms Δ9-THC, Δ8-THC and the acid form THC-A)
CBD (Cannabidiol and the acid form CBD-A)
CBN (Cannabinol)
CBG (Cannabigerol)
CBC (Cannabichromene)
CBL (Cannabicyclol)
CBV (Cannabivarin)
THCV (Tetrahydrocannabivarin)
CBDV (Cannabidivarin)
CBCV (Cannabichromevarin)
CBGV (Cannabigerovarin)
CBGM (Cannabigerol Monomethyl Ether).

Tetrahydrocannabivarin (THCV) is found in certain central Asian and southern African strains of Cannabis.

Cannabidivarin (CBDV) is found in feral Cannabis plants from the northwest Himalayas, and in hashish from Nepal.

Cannabichromene (CBC) is more common in tropical Cannabis varieties.

Thus in numerous embodiments compositions of the invention, apart from THC, CBD and CBN, may also comprise THCA, CBDA, CBG, CBC, CBL, CBV, THCV, CBDV, CBCV, CBGV, CBGM, a derivative, a precursor, or a combination thereof.

Of further relevance to the invention is another group of actives of plant origin, i.e., terpenes (also terpenoids). Terpenes are basic hydrocarbons, whereas terpenoids contain extra functional groups that could be comprised of a range of chemical elements. Terpenoids are flavor and fragrance components Generally Recognized as Safe by the US Food and Drug Administration and other regulatory agencies. Terpenoids are considered potent effectors of animal and human behavior when inhaled from ambient air at serum levels in the single digits ng·mL⁻¹. They are capable of unique therapeutic effects that can contribute to cannabis-based medicinal extracts in increasing their therapeutic index. The nature of phytocannabinoid-terpenoid interaction is still unknown, but it has been acknowledged as synergistic (also referred to an entourage effect) by many examples including treatment of pain, inflammation, depression, anxiety, addiction, epilepsy, cancer, fungal and bacterial infections.

In some embodiments, the terpenes and terpenoids are selected from limonene, myrcene, α-pinene, linalool, β-caryophyllene, caryophyllene oxide, nerolidol and phytol.

Terpenoids share a precursor with phytocannabinoids. For the purpose of present disclosure this types of molecules are referred to herein in terms of classes and individually. Classification of terpenes is based on by the number of isoprene units in the molecule. Monoterpenes consist of two isoprene units and have the molecular formula C₁₀H₁₆. Relevant examples of monoterpenes include limonene, myrcene, linalool or pinene. Sesquiterpenes consist of three isoprene units and have the molecular formula C₁₅H₂₄. Examples of sesquiterpenes include humulene, farnesenes and farnesol.

Various distributions and proportions of terpnoids from these groups in the phyto-derived materials of the invention have been presently exemplified (see Table 3 and Annex A).

Further in this connection, the invention pertains to different types of phyto-derived materials or compositions obtained from distinct types of Cannabis cultivars. The term ‘cultivar’ generally refers to an assemblage of plants selected for desirable characteristics that are maintained during propagation. The presently exemplified Cannabis cultivars are hybrid varieties of C. Sativa and C. Indica, developed to intensify specific characteristics, such as better survival, boosting of flavor, color and smell, or medicinal properties.

It should be noted that the term ‘cultivar’ usually encompasses a number of strains.

Thus, in some embodiments, the invention pertains to three distinct types of cannabis cultivars presently characterized and distinguished on the basis of cannabinoid and terpene content and distribution, and also specific clinical effect on IBD and IBD sub-types. Specifically, the invention pertains to:

i. Phyto-derived compositions obtained from Cannabis strains enriched in a THC in the range of 16-24% (w/w) and relatively low or almost no CBD, an example of which is Erez.

ii. Phyto-derived compositions obtained from Cannabis strains enriched in CBD in the range of 15-16.5% and particularly low THC as 0.8-3.75% (w/w), exemplified by Avidekel.

iii. Phyto-derived material obtained from Cannabis strains having substantially equal ratio of THC:CBD in the range of 6-13% each (w/w), exemplified herein by Midnight.

Further examples of strains of these cultivars (or groups) are shown in Table 3.

Thus, in certain embodiments the invention can be articulated as phyto-derived compositions comprising approximately between 16 and 24% THC and approximately equal or less than 3% CBD (w/w), thus belonging to group (i).

More specifically, the THC content of such compositions can be in a range between at least about 10 and 30%, 11 and 29%, 12 and 28%, 13 and 27%, 14 and 26%, 15 and 25%, 16 and 24%, 17 and 23%, 18 and 22% or about 20% (w/w) or lower. It should be noted that such compositions may comprise a low CBD content in the a range between at least about 0.1 and 1%, 1 and 2%, 2 and 3% or 4 and 5% CBD (w/w). In numerous embodiments, the compositions comprise less than 1% CBD (see Table 3).

In some embodiments, the compositions are further characterized by a CBN content of up to 1% (w/w) or optionally in a range between at least about 0.01 and 1%, 0.1 and 0.9%, 0.2 and 0.8%, 0.3 and 0.7%, 0.4 and 0.6% or about 0.5% (w/w) (see EXAMPLE 1 and Table 4).

In some embodiments, the compositions can be further characterized as phyto-derived THC enriched compositions, wherein CBD constitutes up to about 20% relative to THC and CBN—up to about 7% relative to THC (w/w), or CBD in a range between at least about 1 and 5%, 5 and 10%, 10 and 15%, 15 and 20% relative to THC; and CBN in a range between at least about 10 and 8%, 8 and 6%, 6 and 4%, 4 and 2% or 2 and 1% relative to THC (w/w).

In some embodiments, in the phyto-derived THC enriched compositions of the invention, CBD constitutes up to 4 and 6% THC and CBN in an amount less than 5% relative to THC (w/w).

In other embodiments, the invention provides phyto-derived compositions comprising approximately between 14 and 24% CBD and approximately equal or less than 4% relative to THC (w/w), thus belonging to group (ii) above.

The CBD content in the compositions can be in a range between at least about 10 and 30%, 10 and 20%, 11 and 19%, 12 and 18%, 12.5 and 17.5%, 13 and 17%, 13.5 and 16.5%, 14 and 16%, 14.5 and 15.5%, or about 15% or less (w/w). The compositions can further comprise low THC in a range between at least about 0.1 and 1%, 1 and 2%, 2 and 3% or 4 and 5% relative to THC (w/w). In numerous embodiments, such compositions comprise between about 1 and 2% relative to THC (see Table 3).

The composition may be further characterized by a CBN concentration of up to 1% (w/w), or in a range between at least about 0.01 and 1%, 0.1 and 0.9%, 0.2 and 0.8%, 0.3 and 0.7%, 0.4 and 0.6% or about 0.5% (w/w).

These compositions can be further articulated as phyto-derived CBD enriched compositions, wherein CBD constitutes up to about 600% relative to THC and CBN constitutes up to about 25% relative to THC (w/w), or CBD is in a range between at least about 100 and 200%, 200 and 300%, 300 and 400%, 400 and 500%, 500 and 600%, 600 and 700%, 700 and 800%, or up to 1,000% and more relative to THC, and CBN is in a range between at least about 1 and 5%, 5 and 10%, 10 and 15%, 15 and 20%, 20 and 25%, 25 and 30%, and up to 50% or more, relative to THC (w/w).

In some embodiments in a phyto-derived CBD enriched compositions of the invention, CBD may constitute up to 600% relative to THC and up to CBN 50% relative to THC (w/w).

In still other embodiments, the invention provides phyto-derived compositions comprising approximately equal amounts (or concentrations) of THC and CBD, in a range between at least about 6 and 14% and 6 and 16% (w/w), respectively, thus belonging to group (iii) above.

The CBD or THC content in such compositions can be in a range between at least about 1 and 20%, 2.5 and 17.5%, 5 and 15%, 7.5 and 10% or at least about 12 and 13% (w/w). The compositions are further characterized with CBN content of up to 1% (w/w), or in a range between at least about 0.01 and 1%, 0.1 and 0.9%, 0.2 and 0.8%, 0.3 and 0.7%, 0.4 and 0.6% or about 0.5% (w/w).

These compositions can be further articulated as phyto-derived compositions wherein the amounts of THC and CBD are substantially equal, and wherein CBN constitutes up to about 17% relative to THC (w/w), or in a range between at least about 1 and 5%, 5 and 10, 10 and 15%, 15 and 20% relative to THC (w/w).

In some embodiments, in the phyto-derived compositions comprising substantially equal amounts of THC and CBD, CBN constitutes up to about 7 and 10% relative to THC (w/w).

The role of THC and CBD in the above groups of compositions, in terms of differential therapeutic effects, has been previously discussed. The role of CBN should be perceived in light of the fact that CBN acts as a partial agonists of THC at the CB1 receptors and CB2 receptors. Therefore, various proportions of THC, CBD and CBN in these groups should have direct bearing on their therapeutic properties as reflected in EXAMPLES 2-7.

Still from another point of view, the compositions of group (iii) can be described as compositions wherein the THC:CBD ratio is about 1:1, or substantially 1:1 (w/w), or specifically a ratio in a range between at least about 1.5:1 and 1:1.5 (w/w), and the compositions of groups (i) and (ii) are those wherein said ratio is other than above.

It is meant that compositions herein referred to as enriched in THC can comprise a ratio of THC:CBD in a range between at least about 1.5:1 and 2:1, or 2:1 and 3:1, or 3:1 and 5:1, or 5:1 and 10:1, or 10:1 and 50:1, or 50:1 and 100:1 (w/w), respectively, or more.

In some embodiments, such compositions are referred to as comprising substantially no CBD. The term ‘substantially’ herein refers to a ratio of THC:CBD in a range between at least about 100:1 and 250:1, or 250:1 and 500:1, or 500:1 and 750:1, or 750:1 and 1000:1 (w/w), respectively, or more, or as comprising no measurable CBD.

The compositions of the invention enriched in CBD can comprise a ratio of THC:CBD in a range between at least about 1:1.5-1:2, or 1:2-1:3, or 1:3-1:4, or 1:4-1:5, or further between at least about 1:5-1:10, or 1:10-1:20, 1:20-1:30, 1:30-1:40, 1:40-1:50, 1:50-1:100 (w/w), respectively, or less.

In certain embodiments, such compositions are referred to as comprising substantially only CBD, namely comprising a ratio of THC:CBD in a range between at least about 1:100 and 1:250, or 1:250 and 1:500, or 1:500 and 1:750, or 1:750 and 1:1,000 (w/w), respectively, or less, or as comprising no measurable THC.

In this connection, the terms ‘about’, ‘approximately’, ‘substantially’, which are used interchangeably in this disclosure denote deviation of at least ±10% from the specifically mentioned value of a parameter, e.g., cannabinoid content or distribution (w/w).

Whenever a numerical range is indicated herein, it is meant to include any cited numeral (fractional or integral) within the indicated range. The phrases ‘ranging/ranges between’ a first indicate number and a second indicate number and ‘in the range of’ a first indicate number ‘to’ a second indicate number are used herein interchangeably and are meant to include the first and second indicated numbers and all the fractional and integral numerals in between.

It should be noted that where various embodiments are described by using a given range, the range is given as such merely for convenience and brevity and should not be construed as an inflexible limitation on the scope of the invention. Accordingly, the description of a range should be considered to have specifically disclosed all the possible sub-ranges as well as individual numerical values within that range.

As has been noted in some embodiments, the phyto-derived compositions of the invention can further comprise at least one monoterpene selected from myrcene, limonene and pinene and at least one sesquiterpene selected from caryophyllene, guaiol and farnesene. Presence of these constituents in the compositions of invention with various distribution characteristics of distinct cultivars has been presently exemplified (see Table 3).

It should be further appreciated that in numerous embodiments compositions of the invention are provided in a dosage form adapted for oral administration, or administration by smoking, inhalation and vaporization.

The term ‘composition’ herein encompasses pharmaceutical compositions, which may be presented in unit dosage forms using techniques well known in the pharmaceutical industry. In the same way, the terms ‘carrier’, ‘buffer’, ‘excipient’ herein encompass ‘pharmaceutically acceptable carriers’, for example, vehicles, adjuvants, excipients, or diluents, well-known to those who are skilled in the art. A pharmaceutically acceptable carrier is usually chemically inert and has no detrimental side effects or toxicity.

In connection with oral dosage form, e.g., oil extracts exemplified herein by compositions derived from Avidekel, in numerous embodiments such compositions can further comprise at least one drug or therapeutic agent relevant to IBD. Alternatively, in numerous other embodiments, therapeutic methods using compositions of the invention can comprise concomitant administering of at least one drug relevant to IBD.

In some embodiments, the therapeutic agents or drugs belong to the groups of anti-inflammatory, anti-nociceptive, antibiotic, antiemetic, anti-diarrheal drugs, or any combination thereof.

Notable examples of therapeutic agents relevant IBD include, although not limited to:
Anti-inflammatory drugs, predominantly mesalazine (INN, BAN), also known as mesalamine (USAN) or 5-aminosalicylic acid (5-ASA), available in several oral formulations (brand names Asacol, Delzicol, Asacol HD, Pentasa, Dipentum, Colazal, Apriso, and Lialda);
Corticosteroid drugs, including cortisone, hydrocortisone, prednisone and budesonide, available, among others, in oral formulations and by injection;
Biological drugs, predominantly monoclonal antibodies, including infliximab (INN; brand names Remicade, Remsima, Inflectra) and adalimumab (INN; brand names Humira and Exemptia), targeting tumor necrosis factor alpha (TNF-α);
Immunosuppressive antimetabolites, including azathioprine (INN; brand name Imuran), methotrexate (INN; brand names Rheumatrex, Trexall, Otrexup, Rasuvo) and cyclosporine (INN; brand name Sandimmune), available in oral formulations.

In some embodiments, compositions of the invention, per se, or in combination with other drugs, are intended to treat, alleviate or reduce IBD, or at least one symptom of IBD, as revealed by measuring a reduction of at least one of a score according to Disease Activity Index (DAI) and/or Simple Endoscopic Score for Crohn's Disease (SES-CD), a level of an inflammatory marker in blood and/or a fecal sample, and/or an improvement of at least one of weight, self-reporting on pain, bowel movement and quality of life (see EXAMPLES 2-7).

As has been demonstrated herein, using the above measurements of improvement, certain compositions of the invention are capable of treating, reducing and alleviating more than one symptom of Crohn's disease. In some embodiments, such compositions are referred to as CBD enriched compositions (see EXAMPLE 2).

As has been further demonstrated, certain other compositions of the invention are capable of treating, reducing and alleviating at least one symptom of colitis. In some embodiments, such compositions are referred to as THC enriched compositions (see EXAMPLE 3).

Certain examples of THC enriched compositions of the invention have been presently demonstrated. Those include, although not limited to, those derived from at least one Cannabis strain herein designated herein as Erez, Alaska, Eran-Almog, Dorit, Omer, Shira, Or, Zohar, Barak, Tal or Jasmine.

Examples of CBD enriched compositions include, although not limited to, compositions derived from at least one Cannabis strain herein designated herein as Avidekel or Rephael.

Example of compositions of the invention wherein THC and CBD are approximately equal include, although not limited to, those derived from at least one cannabis strain herein designated as Midnight, Elna or Mango.

For the purpose of certain embodiments compositions derived from THC enriched strains or strains wherein THC and CBD are equal, are provided in a dosage form of a dry plant material adapted for smoking, inhalation or vaporization. For the purpose of other embodiments, compositions derived from CBD enriched strains are provided in an oral dosage form, e.g., an oil extract.

It should be appreciated that the compositions of the invention may further comprise various additives, being natural or synthetic substance formulated alongside an active ingredient for the purpose of long-term stabilization, bulking up solid formulations, or to confer a therapeutic enhancement on an active ingredient in the final dosage form, such as facilitating drug absorption, reducing viscosity, or enhancing solubility. Types of additives include: antiadherents (e.g. magnesium stearate), binders (e.g. saccharides, gelatin, synthetic polymers), coating agents (e.g. cellulose ethers), colorants (e.g. titanium oxide), disintegrants (e.g. crosslinked polymers), flavors, glidants or lubricants (e.g. talk, vegetable stearin), preservatives (e.g. antioxidants), sorbents (e.g. desiccants), sweeteners, vehicles (e.g. petrolatum and oils).

In some embodiments, the cannabis-based compositions of the invention, being in some embodiments, oral dosage forms as described above, comprise natural oils, e.g. olive oil.

Compositions of the invention may further comprise other phyto-derived compounds, i.e., nitrogenous compounds, amino acids, proteins, enzymes, glycoproteins, hydrocarbons, alcohols, aldehydes, ketones, fatty acids, esters and lactones, steroids, terpenes, non-cannabinoid phenols, flavonoids, vitamins and pigments, relative abundance of which differs between Cannabis varieties. Some compounds (e.g. terpenes, flavonoids) also act as antioxidants, anti-anxiety, anti-inflammatory, anti-bacterial, anti-neoplastic agents.

It is another aspect of the invention to provide methods for treating, alleviating or reducing at least one symptom of IBD in a patient in need thereof, said method comprising administering to the patient at least one phyto-derived composition comprising at least one cannabinoid and at least one terpene, wherein
(a) said composition is derived from at least one of a cannabis plant enriched in THC, a cannabis plant enriched in CBD, a cannabis plant wherein the amounts of THC and CBD are substantially equal,
(b) at least one of the cannabinoid is selected from THC, CBD and CBN, and
(c) said at least one terpene is selected from monoterpenes and sesquiterpenes.

It should be noted in this connection that methods of the invention are further intended for treating, alleviating or reducing partial symptoms of IBD, referred to herein as ‘at least one symptom’.

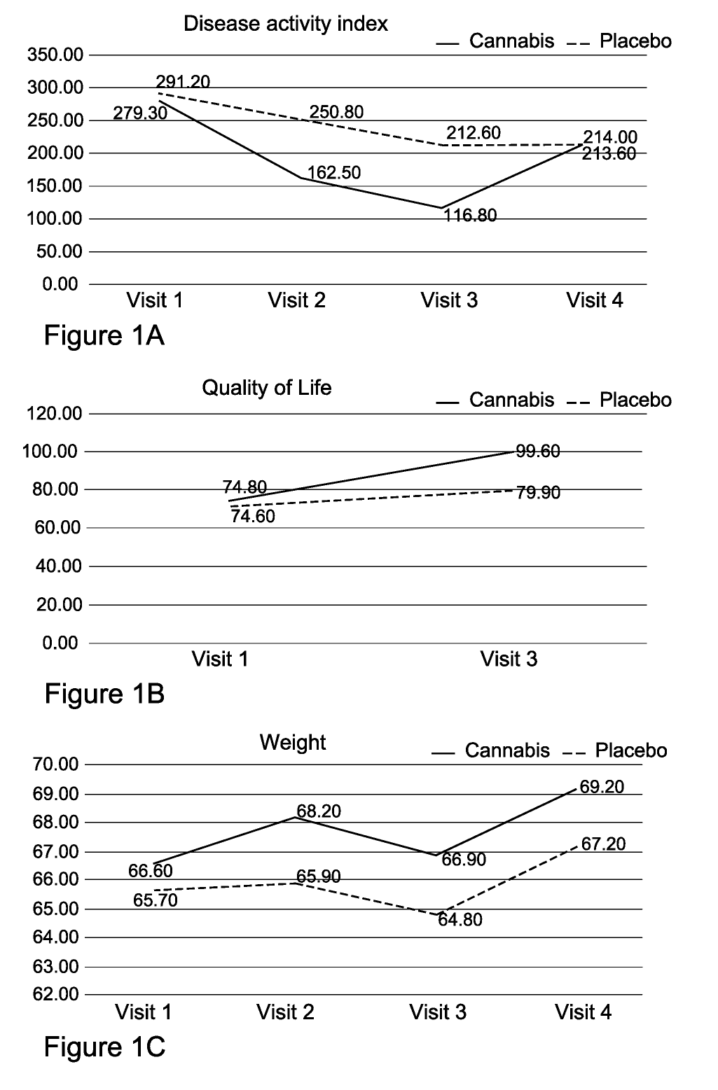

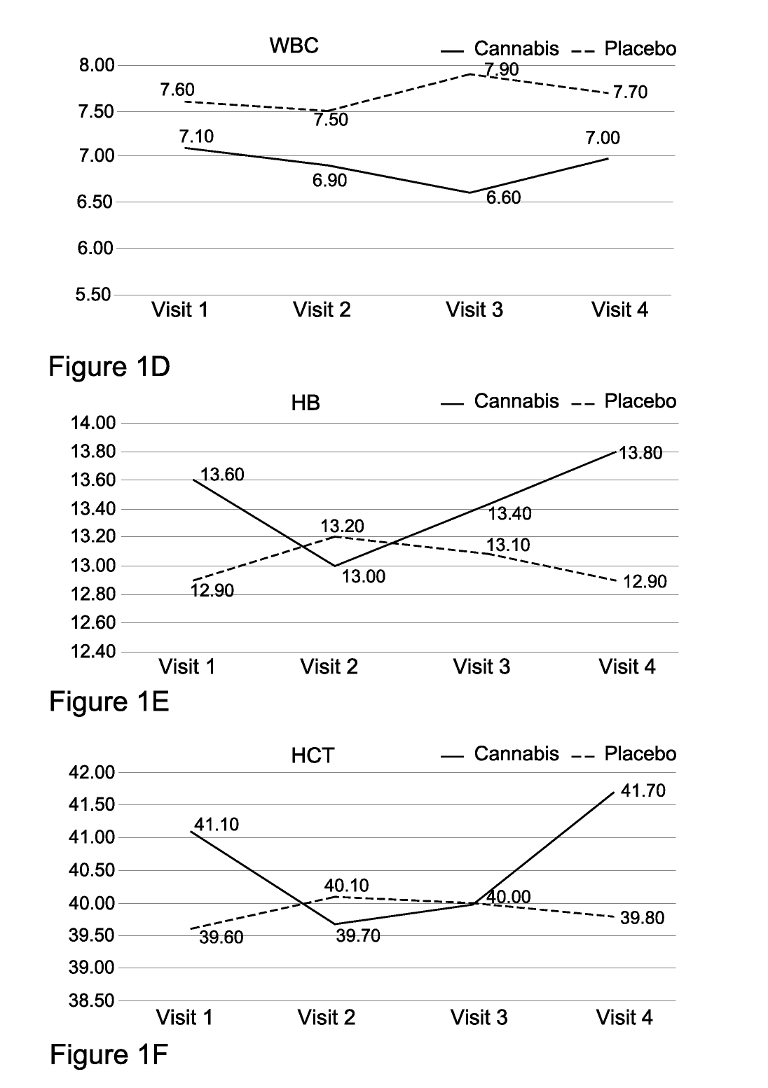

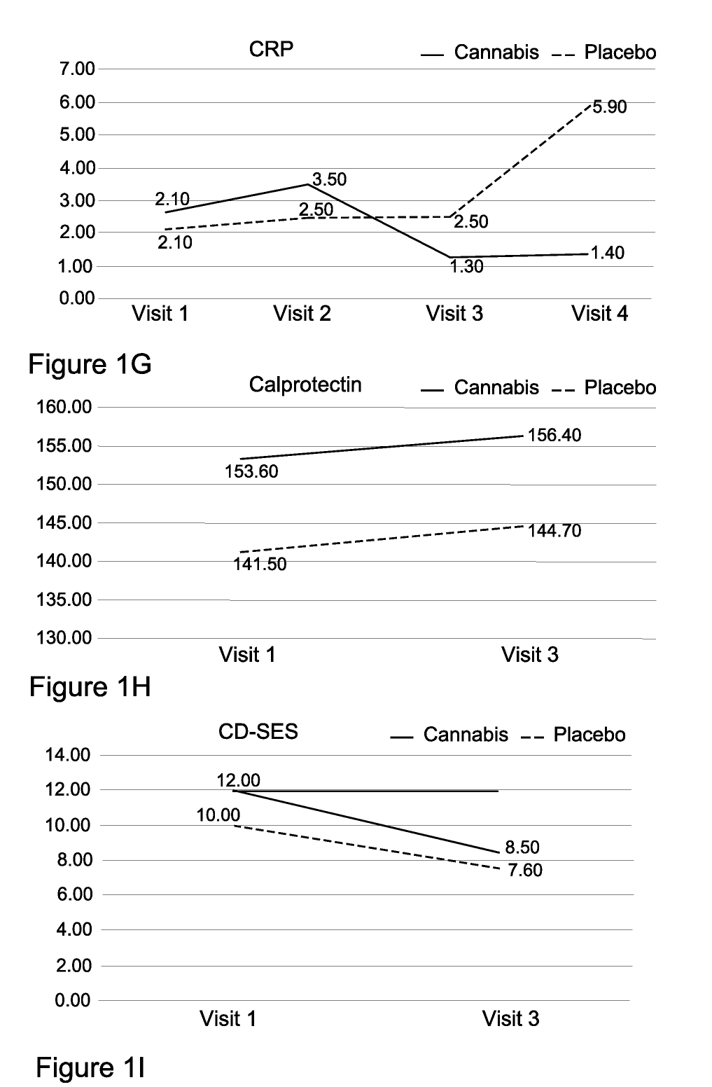

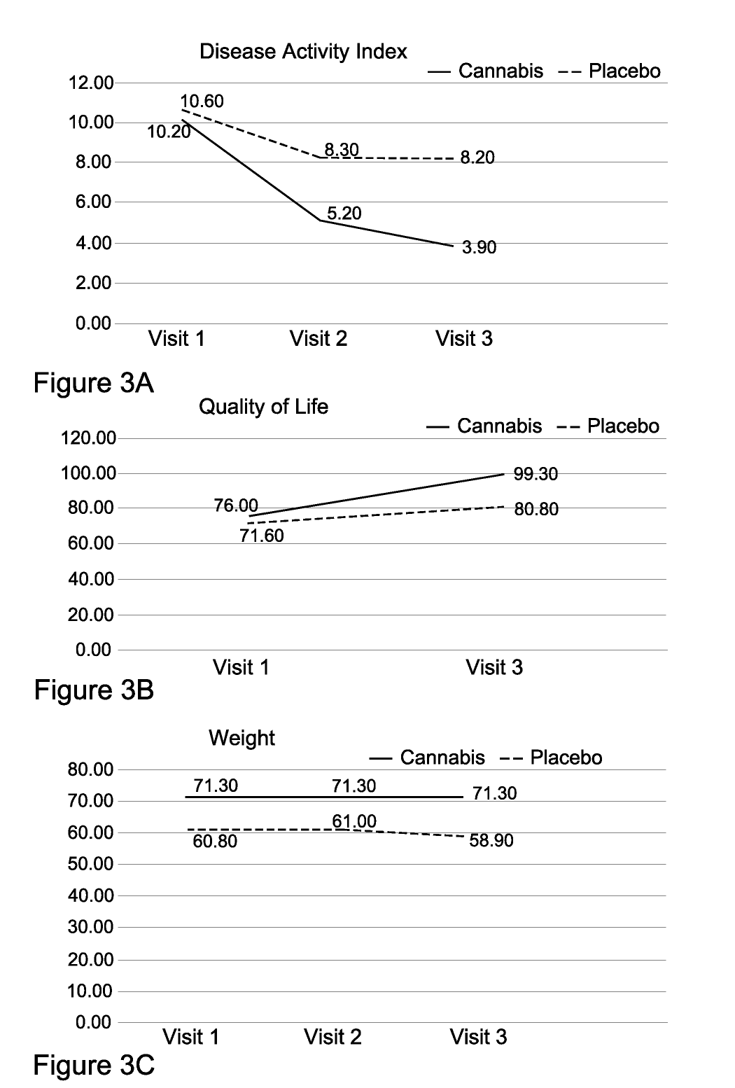

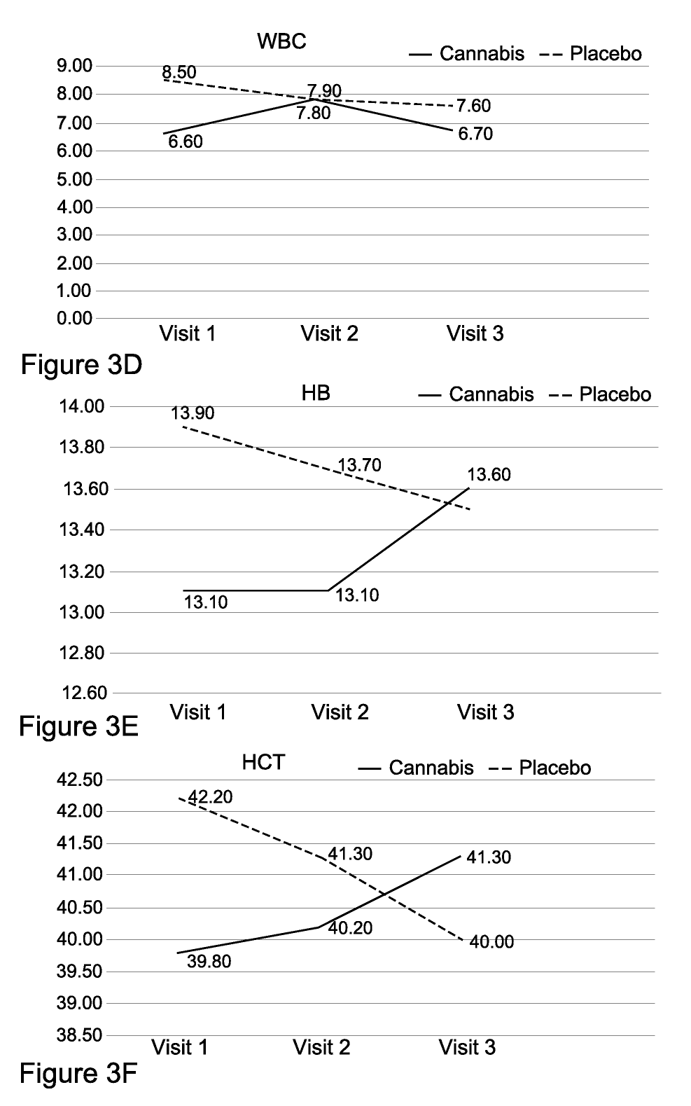

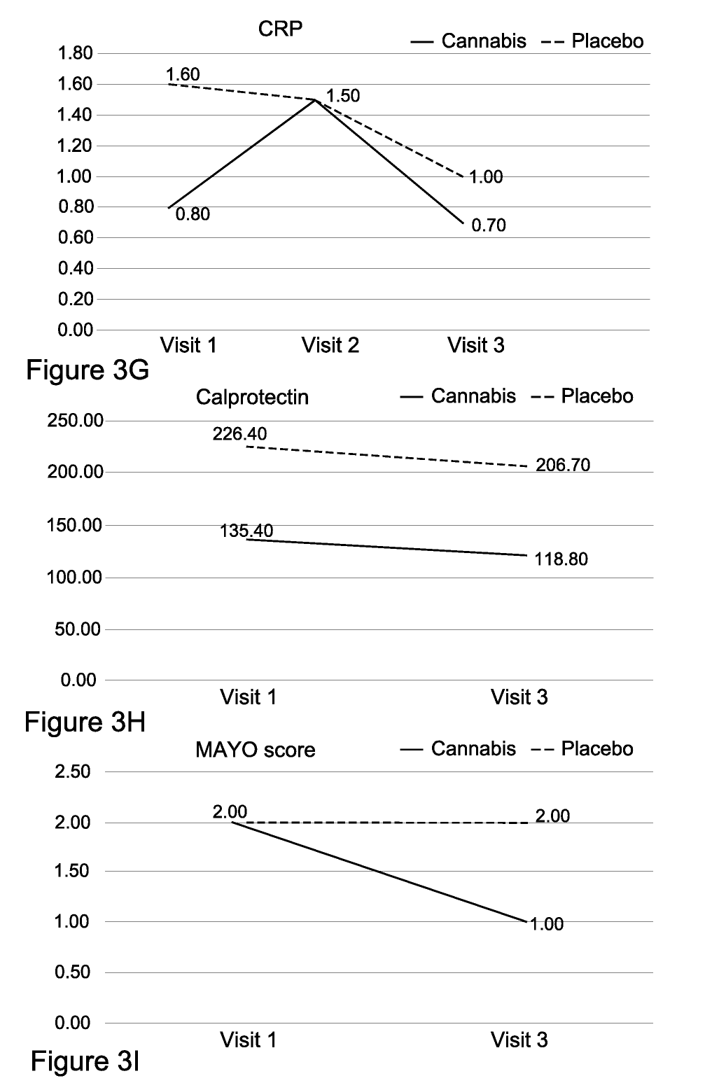

In some embodiments, therapeutic effects of methods of the invention become apparent by measuring a reduction of at least one of a score according to Disease Activity Index (DAI) and/or Simple Endoscopic Score for Crohn's Disease (SES-CD), a level of an inflammatory marker in blood and/or a fecal sample, and/or an improvement of at least one of weight, self-reporting on pain, bowel movement, quality of life. Applicability of such measurements and tests has been presently exemplified (see EXAMPLES 2-7 and FIGS. 1A-1I, and 3A-3I).

In some embodiments, methods of the invention apply to patients suffering from Crohn's disease or colitis.

In some embodiments, above methods further comprise concomitant administering of one or more additional drug relevant to IBD. The term ‘concomitant’ administering or co-administering encompasses administering at the same time (simultaneous) and in succession. Consecutive administering refers herein to administration of one or more compositions of the invention, or one or more compositions of the invention and state-of-the-art pharmaceutical compositions within a certain time period, such as a span of 72 hours, 48 hours, 24 hours, 12 hours, 6 hours, 3 hours, 2 hours, 1 hour, or less than 1 hour, or at the same time. Drugs that are relevant to IBD and related conditions have been described above.

It should be appreciated that compositions and methods of the invention are applicable to various patients of all ages and both genders. IBD has been reported in all age groups, but adolescents and young adults between the ages of 15 and 35 are considered to be most susceptible, 10% of those afflicted are under the age of 18. Another peak in the occurrence of IBD is after age 50. IBD is considered to be more prevalent in females than males. Methods of the invention are applicable to all age groups for being non-invasive. In numerous embodiments, the phyto-derived compositions are administered orally or by inhalation, vaporization, or a combination thereof, and therefore can be applicable to children or elderly patients alike. In certain embodiments, methods of the invention involve administering by smoking alone or in combination with the above.

In some embodiments, compositions and methods of the invention can apply to patients considered non-responders to conventional therapies, e.g., adalimumab (Humira).

The terms ‘therapeutic dose’ or ‘therapeutically effective dose’, wherein herein are interchangeable, relate to doses of a composition of the invention, in any dosage form, that produces improvement of at least one symptom of IBD, measured as above. In this sense, the therapeutic effect is also a pharmacodymanic effect.

In certain embodiments, said improvement of IBD is at least 5%, 10%, 15%, 20% improvement, or at least 25%, or at least 50%, or at least 75%, or at least 100% improvement. The improvement can involve an improvement in more than one symptom, in terms of severity, frequency or recurrence and use of concurrent medication, etc.

A therapeutically effective amount (also pharmacologically or pharmaceutically or physiologically effective amount) means herein an amount of active agent (phyto-derived compositions of the invention) in a pharmaceutical composition that is needed to provide a desired level of active agent in the bloodstream or at a target organ of to provide an anticipated physiological response. The precise amount will depend upon numerous factors, e.g. type of an agent, activity of a composition, intended patient use (e.g. number of doses per day), patient considerations, and others, which can readily be determined by one skilled in the art. An effective amount of an agent can be administered in one administration, or through multiple administrations of an amount that total an effective amount, preferably within a 24-hour period. It can be determined using standard clinical procedures for determining appropriate amounts and timing of administration. It is understood that the effective amount can be the result of empirical and/or individualized (case-by-case) determination on the part of the treating health care professional and/or individual.

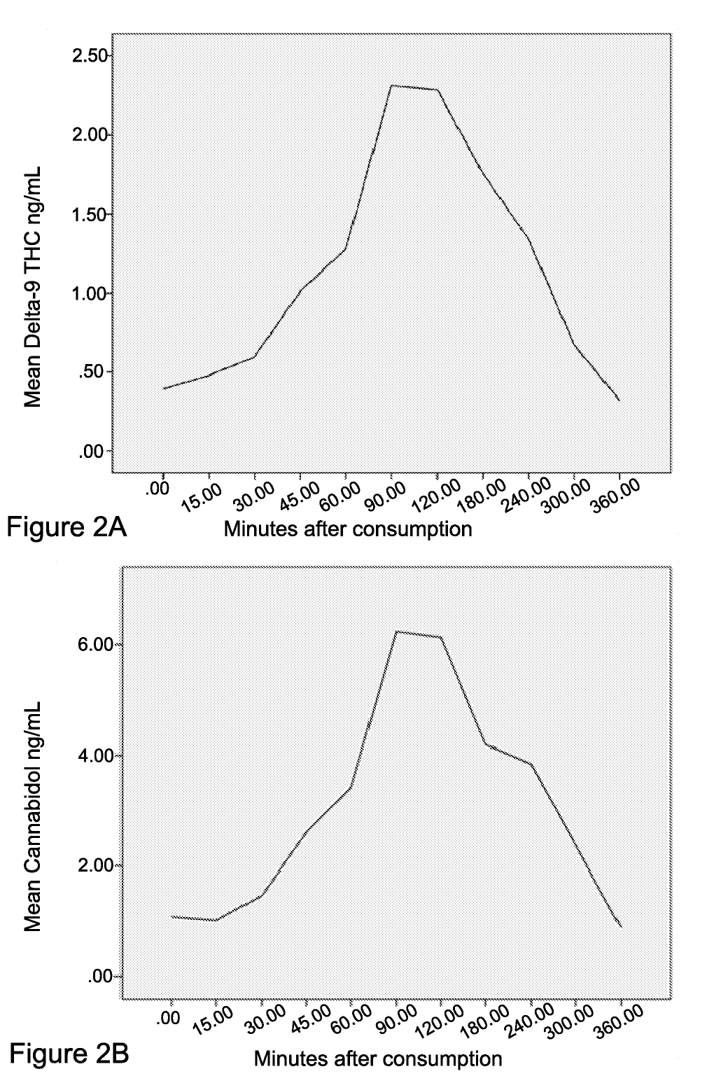

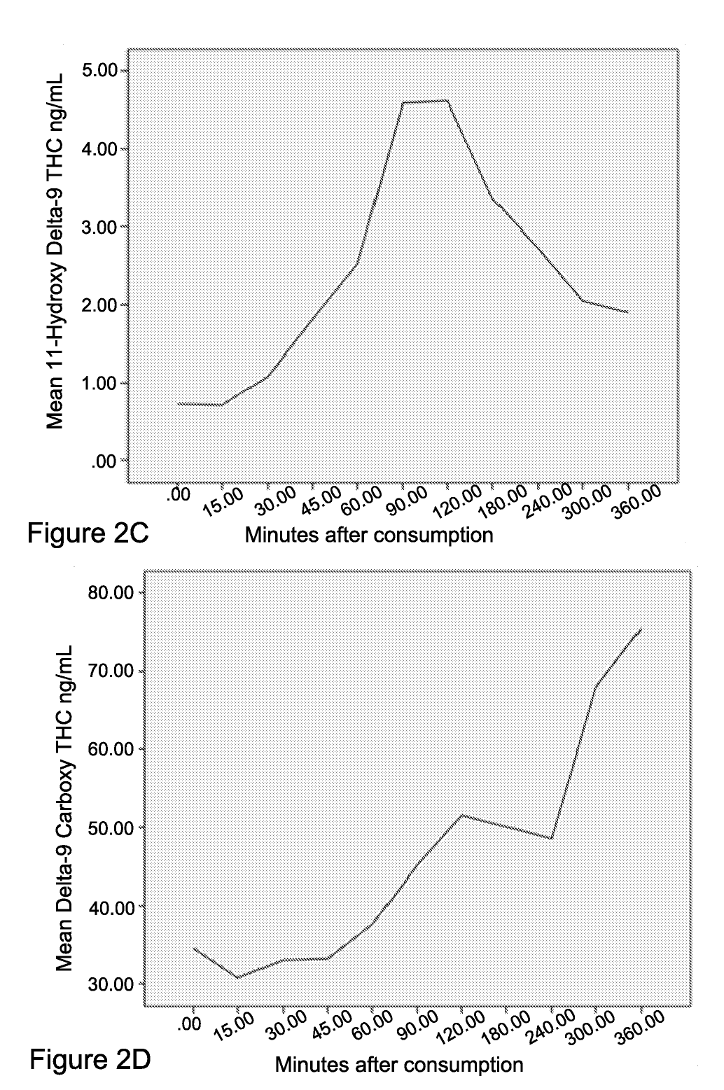

In this connection, pharmacokinetic profiles of certain phyto-derived compositions of the invention have been presently demonstrated, specifically Avidekel derived CBD enriched oil extracts (see EXAMPLE 2 and FIGS. 2A-2D).

It is another feature of the invention to provide compositions and methods for immediate and/or prolonged alleviation, reduction or treatment of complete or partial symptoms of IBD. The terms ‘immediate’ and ‘prolonged’ herein refer to an onset and a duration of therapeutic effects of the composition of the invention, defined by improvement of said symptom(s) according to previously detailed measurements and specific disease indices.

Under the term ‘immediate’ is meant an onset of a therapeutic effect within about 1 and 30 min after administering a composition of the invention, or in a range of between at least about 1 and 30 min, 1 and 20 min, 1 and 15 min, 1 and 10 min, 1 and 5 min, or less, with a duration of at least about 1 and 30 min, 1 and 40 min, 1 and 50 min, 1 and 60 min, and up to 2 hours, or more, the duration being further depended on administered dose and administration route.

In some embodiments, methods and compositions of the invention apply to immediate alleviation of IBD symptoms, specifically those involving administering of phyto-derived compositions enriched in THC or wherein THC and CBD amounts are substantially equal. Immediate effects of such compositions have been presently demonstrated (see EXAMPLE 3)

In certain embodiments, the methods involve administering of compositions comprising approximately 16-24% THC and approximately equal or less than 3% CBD or approximately 6-14% THC and 6-16% CBD (w/w). In further embodiments, the methods involve administering compositions further comprising up to about 1% CBN (w/w).

In further embodiments, CBD comprised in the compositions constitutes up to about 20% relative to THC and CBN—constitutes up to about 7% relative to THC (w/w), or for compositions wherein THC and CBD are substantially equal, CBN constitutes up to about 17% relative to THC (w/w).

In some embodiments, the methods involve administering of a composition of the invention by smoking, inhalation, vaporization or a combination thereof.

In certain embodiments, such methods involve administering of at least one composition derived from at least one cannabis strain herein designated Erez, Alaska, Eran-Almog, Dorit, Omer, Shira, Or, Zohar, Barak, Tal, Jasmine, Midnight, Elna or Mango.

It is a further feature of the invention to provide a method for treating colitis by administering a THC-enriched composition derived from a cannabis strain herein designated Erez, which is administered by smoking, inhalation, vaporization or a combination thereof.

In other embodiments, methods and compositions of the invention apply to prolonged alleviation of IBD symptoms, specifically those involving administering of phyto-derived compositions enriched in CBD. Under the term ‘prolonged’ is meant an onset of a therapeutic effect more than 30 min after administering the compositions of the invention, or in a range of between 30 and 40 min, 30 and 50 min, 30 and 60 min, 30 and 120 min or more, with a duration of at least about 1 and 2 hours, 1 and 3 hours 1 and 4 hours, 1 and 5 hour, 1 and 6 hours, 1 and 10 hours, 1 and 20 hours, 1 and 30 hours or more, the duration being further depended on administered dose and administration route.

In some embodiments, such methods involve administering of compositions comprising approximately 14-24% CBD and approximately equal or less than 4% THC (w/w). In some embodiments the methods involve administering compositions further comprising up to about 1% CBN (w/w). In further embodiments, CBD comprised in these compositions constitutes up to about relative to 600% THC (w/w), and CBN constitutes up to about 50% relative to THC (w/w).

In some embodiments, the above methods involve oral administration of the compositions. In certain embodiments, such methods involve administering of at least one cannabis strain herein designated Avidekel or Rephael.

It is another specific feature of the invention to provide a method for treating Crohn's disease by oral administering a CBD-enriched composition derived from a cannabis strain herein designated Avidekel.

It is yet another important aspect of the invention to provide methods for long-term treatment and management of IBD and related conditions. Specifically, such methods involve a combination therapy comprising administering to a patient with IBD

(i) at least one composition derived from a cannabis plant enriched in THC or a cannabis plant wherein the amounts of THC and CBD are substantially equal, and

(ii) at least one composition derived from a cannabis plant enriched in CBD.

In some embodiments, administrations (i) and (ii) are carried out in a daily regimen in succession.

In further embodiments, administration (i) is carried out before sleep, and administration (ii) is carried out during waking hours.

In certain embodiments the composition administered in step (i) comprises between approximately 16 and 24% THC and approximately equal or less than 3% CBD, or between approximately 6 and 14% THC and 6 and 16% CBD (w/w), and the composition administered in step (ii) comprises between approximately 14 and 24% CBD and approximately equal or less than 4% THC (w/w).

In further embodiments, the combination therapy involves administering compositions further comprising up to about 1% CBN (w/w).

In still further embodiments, CBD comprised in compositions utilized in step (i) constitutes up to about 20% relative to THC, and CBN constitutes up to about 7% relative to THC (w/w), or for compositions wherein THC and CBD are substantially in equal amounts, CBN constitutes up to 17% relative to THC (w/w), and for compositions in step (ii) CBD constitutes up to about 600% relative to THC (w/w) and CBN constitutes up to about 50% relative to THC (w/w).

In certain embodiments, the methods involve administering in step (i) compositions derived from at least one cannabis strain herein designated as Erez, Alaska, Eran-Almog, Dorit, Omer, Shira, Or, Zohar, Barak, Tal, Jasmine, Midnight, Elna or Mango, and in step (ii) compositions derived from at least one cannabis strain herein designated as Avidekel or Rephael.

In further embodiments, compositions administered in step (i) are administered by smoking, inhalation, vaporization or a combination thereof, and compositions administrated in step (ii) are orally administered.

Combination therapies have been investigated in detail in EXAMPLE 7. Surprising benefits of a combination therapy using THC enriched and CBD enriched compositions in succession have been demonstrated by significant improvement of disease indices, significantly lower number and severity of adverse events, and improvement in general quality of life compared to monotherapies using THC enriched or CBD enriched compositions.

Most surprisingly, such combination therapies proved to be more beneficial than monotherapies using THC and CBD in combination, when administered in the same composition (i.e., Midnight).

Moreover, combination therapies proved to be more efficient in the management of pain for which THC enriched compositions have been considered, so far, more effective.

With respect to the presently exemplified dosage forms, THC enriched compositions of the invention in the form of cigarettes or those comprising equal THC and CBD are intended for immediate relief of IBD symptoms and/or also colitis. The absolute amount of THC delivered in the smoke varies widely and has been estimated at between 20 and 70%, the remainder being lost through combustion or side-stream smoke. Tolerable doses of the THC in the form of cigarettes can reach up to between 60 and 70 mg per day.

In terms of daily doses, in certain embodiments such cigarettes are consumed daily, preferably before sleep, or with the onset of symptom(s), as one or two cigarettes per day, or more, as an occasional, a periodic or a continuous treatment. In terms of oral dosage forms, specifically oil extracts enriched in CBD, these are intended for prolonged alleviation of symptom(s) of IBD and/or Crohn's disease. This type of compositions is consumed in the form of drops. A drop of Avidekel oil, for example, 0.04 ml in volume has been estimated as having about 6 mg CBD and 1.5 mg THC.

In some embodiments, a single oral dosage form comprises about up to 14-15 drops, or in the range of 1-2, 2-4, 4-6, 6-8, 8-10, 10-12, 12-14 drops or more, with average CBD/THC content per administration in a range between at least about 10-100 mg, 10-80 mg, 10-70 mg, 10-60 mg, or 10-50 mg CBD, or 15-45 mg, 20-40 mg, 25-35 mg, or about 30 mg CBD, and 5-8 mg, 5.5-7.5 mg, 6-7 mg, or about 6.5 mg THC.

It should be appreciated that in certain embodiments a single oral dosage form comprises an average THC content of less than 5 mg per administration.

In terms of daily doses, to obtain prolonged effects said oral dosage forms are administered at least once a day, two times a day or three times a day or more, with average daily doses in a range between at least about 50-100 mg, 100-150 mg, 150-200 mg, 200-250 mg CBD, or more, with maximal daily doses up to at least about 300-500 mg CBD per day, and 15-25, 16-24, 17-23, 18-22, 19-20 mg THC, or less.

Further, in certain embodiments to obtain prolonged and sustainable effects said CBD enriched oral dosage forms are administered continuously for a period of at least about up to 4, 5, 6, 7, 8, 9, 10, 11, 12 weeks, months and years, or the entire period of persistence of symptom(s).

In terms of daily regimen, such oral dosage forms are taken during the day as periodic or continuous treatment.

In one of its further aspects, the invention provides use of phyto-derived compositions of the invention for the manufacture of a medicament for the treatment or alleviation or a reduction of at least one symptom of IBD.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

As will be apparent to those of skill in the art upon reading this disclosure, each of the individual embodiments described herein has discrete components and features which may be readily separated from or combined with the features of any of the other several embodiments without departing from the scope or spirit of the present disclosure. Any recited method can be carried out in the order of events recited or in any other order that is logically possible.

The examples are representative of techniques employed by the inventors in carrying out aspects of the invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Materials and Methods

1. Biochemical Analyses of Phyto-Derived Compositions of the Invention

Cannabinoid and terpene contend was determined using standard procedures for gas chromatography-mass spectrometry (GC-MS) analysis. In brief, dry plant-derived material were powdered, extracted with an organic solvent (n-hexane), filtered, and samples (1 μL) were loaded on GC-MS (Hewlett Packard G 1800B GCD system with a HP-5971 gas chromatograph with electron ionization detector). Compounds of interest were identified by comparison with standards, retention times, Kovats indices, and available libraries (software GCD Plus Chemstation). Ratios of specific cannabinoids (THC, CBD) were determined relative to total cannabinoid content, and ratios of specific terpenes ratios—to main terpene (100%).

2. Preparation of Oil Extracts from Flower-Derived Material

Oil extracts were prepared from Avidekel strain in the presence of olive oil using previously described procedures, i.e., CO2 extraction processes or solvent evaporation using ethanol. Cannabinoid content of oil preparations was determined using LC-MS or HPLC by means of standard procedures. Batches of oil extracts were monitored according to ISO9001 and HACCP standards of quality management.

3. Dose Titration in Clinical Trials

Each patient participating in the clinical studies was subjected to titration of individual dose. The duration of titration period was approximately 3 weeks, wherein the initial dose and/or dose regimen (number of administrations per day) were gradually incremented to achieve maximal clinical effect with minimal adverse reactions (evaluated according to mental status, behavioral and psychological symptoms of dementia (BPSD), decline in motor function and stability, significant changes in blood pressure, sugar levels, respiration rates, pulse). Daily doses did not exceeded 400 mg active cannabinoids, CBD and THC.

4. Prospective Clinical Study in Patients with Crohn's Disease

Patients were randomly assigned to receive either Avidekel oil extract comprising THC 4% and CBD 16% (THC:CBD ratio 1:4), or placebo comprising olive oil with chlorophyll. Both patients and investigators were blinded to the assignment procedure. Patients were subjected to follow up period of 8 weeks of treatment and wash out period of additional 2 weeks. The follow up data at baseline and weeks 2, 8, and 10 included: clinical interview, physical examination, assessment of disease activity (CDAI), and blood tests (complete blood count, liver and kidney function, C-reactive protein (CRP) marker for inflammation), SES colonoscopy, calprotectin test for direct intestinal inflammation, and measurement of physical and mental health status using and reporting on side effects using a standardized Quality of Life (SF-36) questionnaire.

5. Pharmacokinetic Studies in Patients with Crohn's Disease

Patients with Crohn's disease (N=7) participating in the prospective clinical trial. Blood samples were withdrawn at time 0 and after administration of Avidekel oil extract, a sublingual dose 4 drops. Blood samples were withdrawn in intervals of 15, 30, 45, 60 and 90 min., and 2, 3, 4, 5, and 6 h., and stored at −70° C. until analysis at NMS Labs using LC-MS/MS. The analysis related to two main cannabinoids and two metabolites, including THC (Δ⁹-THC), CBD, 11-Hydroxy Δ⁹-THC (active metabolite) and Δ⁹ Carboxy THC (inactive metabolite).

6. Clinical Study in Patients with Ulcerative Colitis

Patients were randomly assigned to receive either Erez in the form of cigarettes containing 1 gr flower-derived material comprising THC 23% or placebo cigarettes. Patients were subjected to follow up period of 8 weeks of treatment and wash out period of additional 2 weeks. The follow up data at baseline and weeks 2, 8, and 10 included: clinical interview, physical examination, assessment of disease activity (DAI), and blood tests (complete blood count, liver and kidney function, C-reactive protein (CRP) marker for inflammation), SES colonoscopy, calprotectin test for direct intestinal inflammation, and measurement of physical and mental health status using and reporting on side effects using a standardized Quality of Life (SF-36) questionnaire.

7. Retrospective Study of Patients Receiving Mono- or Combination Therapies

Patients' data was retrieved from the company database including demographic and clinical data and clinical follow up on more than 1800 patients with various clinical conditions who received phyto-derived cannabinoid compositions of the invention under specified regimens. Data on patients with clinical diagnosis of IBD, Crohn's disease and colitis, was selected for this study (N=291). Data included clinical anamnesis, physical examination and clinical evaluation of IBD in relating to DAI clinical severity score, biochemical tests for indices of blood inflammation, faecal calprotectin, and colonoscopy using MAYO and CD-SES scores. A further evaluation relating indices of patient's life-style, quality of life, personal preferences, etc, was retrieved from QOL (SF-36) questionnaires. Additional data related to reports on the presence of adverse events by study physicians, during at least three following visits, during the first month and the first year. A group of patients had more than 6 months experience with phyto-derived compositions of the invention (N=142). Data was subjected to relevant variance analyses (e.g., T-test ANOVA, Mann-Whitney) to reveal associations with beneficial treatment outcomes (p<0.05 was considered statistically significant).

### Example 1

1a. Cannabinoid and Terpene Profiles in the Compositions of the Invention

Table 3 shows relative content of cannabinoids and terpenes in phyto-derived compostions of the invention, including the two main cannabinoids (THC and CBD ratios) and a number of terpenes of monoterpenes (myrcene, limonene, β-pinene) and sesquiterpenes (β-caryophyllene, guaiol, β-farnesene) classes. The complete terpene data is provided in Annex A.

TABLE 3
Profiles of representative cannabinoids and terpenes in phyto-derived material

| Strain | THC | CBD | Myrcene | Limonene | β-pipene | β-caryophyllene | Guaiol | β-farnesene |
|---|---|---|---|---|---|---|---|---|
| (name) | (%) | (%) | (%) | (%) | (%) | (%) | (%) | (%) |
| Avidekel | 1.1-2 | 14.5-16.3 | 100 | 17.4 | 21.3 | 37.6 | 45.5 | 13.04 |
| Barak | 18-20 | <0.1 | 100 | 16.6 | 16.9 | 54.2 | 24.9 | 19.1 |
| Erez | 20-24 | <0.1 | 100 | 15.4 | 15.8 | 83.7 | 41.2 | <1 |
| Jasmin | 14-16 | <0.1 | 100 | 20.4 | 26.3 | 15.4 | 13.6 | 1.5 |
| Tal | 18-20 | <0.1 | 100 | 18.0 | 14.9 | 50.6 | 21.2 | <1 |
| Shira | 18-20 | <0.1 | 100 | 32.7 | 29.7 | 80.5 | <1 | 2.9 |
| Or | 20-24 | <0.1 | 35.6 | 2.1 | 52.2 | 84.4 | 28.0 | 23.7 |
| Refael | 1.1-2 | 15-17 | 66.7 | 22.6 | 8.8 | 100 | 50.6 | 6.0 |
| El-na | 10-12 | 4-6.5 | 93.1 | 26.1 | 42.1 | 96.1 | 86.3 | 9.3 |
| Alaska | 20-22 | <0.1 | 50.2 | 46.0 | 45.7 | 65.4 | 86.0 | 6.3 |
| Eran-Almog | 24-28 | <0.1 | 68.3 | 41.8 | 32.0 | 100 | 66.0 | 31.9 |
| Midnight | 10-13 | 8-12.5 | 30.5 | 17.1 | 6.3 | 100 | 64.1 | 31.0 |
| Dorit | 18-20 | <0.1 | 17.9 | 4.4 | 9.5 | 100 | 34.1 | 26.0 |
| Mango | 6-9 | 6-9 | 67.7 | <1 | 13.3 | 100 | 5.2 | 30.7 |
| Omer | 20-24 | <0.1 | 100 | 8.7 | 16.8 | 69.6 | 32.0 | 26.6 |

Additional data on relative content cannabinoids in phyto-derived material is presented below:
Avidekel was identified with a CBD-enriched content, i.e., 14-22% CBD, 0-2% THC and 0-1% CBN (w/w) in a flower-derived material (w/w);
Rafael, similar to Avidekel, was identified with 16-24% CBD, 0-2% THC 0-1% CBN (w/w) regarding the same;
Erez was identified with a THC-enriched content, i.e., 16-24% THC, 0-2.5% CBD and 0-1% CBN (w/w);
Alaska, Eran-Almog, Dorit, Omer, Shira, Or (and a more recent strain Zohar) showed similar profiles;
Midnight, in contrast, were identified with equal or almost equal THC and CBD, i.e., 8-16% CBD, 6-14% THC, 0-1% CBN (w/w).
Mango was also identified with equal THC and CBD in the range of 6-9% (w/w).

This data showed that phyto-derived materials from specific strains were identified with particular relative content (ratio) of cannabinoids and terpenes. Specifically, with regard to the relative content of THC and CBD, the phyto-derived material of the invention were grouped into three main categories:

i—THC enriched material, e.g., derived from Erez, Alaska, Eran-Almog;

ii—CBD enriched material, e.g., derived from Avidekel, Refael; and

iii—material derived from strains wherein THC and CBD content is approximately equal, e.g., Midnight.

With regard to terpenes, phyto-derived materials from specific strains were identified with specific relative content of monoterpenes and sesquiterpenes, and specific terpenes of these classes. Significant differences were observed in relative content of myrcene, limonene (monoterpenes), e.g., Dorit and Avidekel, and of β-caryophyllene, guaiol, e.g., Midnight and Jasmin.

These biochemical properties of phyto-derived composition of the invention were further related to differential effects on partial symptoms of IBD and general alleviation of IBD condition.

1b. Relative Content of Main Cannabinoids in Oil Extract of Avidekel

Table 4 shows main cannabinoid profiles in oil extracts of Avidekel, CBD enriched. material, as determined in two independent experiments using HPLC.

TABLE 4
HPLC analysis of Avidekel oil extracts.

| Assay by | Experiment I | Experiment II |
|---|---|---|
| HPLC | (%) | (%) |
| CBDA | <0.1% | <0.1% |
| CBG | 0.74 | 0.41 |
| ¹Cannabidiol (CBD) | 16.34 | 15.38 |
| ³Cannabinol (CBN) | 0.12 | <0.1% |
| ²Tetrahydrocannabinol (THC) | 4.01 | 3.93 |
| CBC | 0.8 | 0.74 |
| THCA | ND | <0.1% |

¹Cannabidiol: 2-[(1R,6R)-6-isopropenyl-3-1-yl]-5-pentylbenzene-1,3-diol.
²Tetrahydrocannabinol: (−)-(6aR,10aR)-6,6,9-Trimethyl-3-methylcyclohex-2-en-pentyl-6a,7,8,10α-tetrahydro-6H-benzo[c]chromen-1-ol.
³Cannabinol: 6,6,9-Trimethyl-3-pentyl-benzo[c]chromen-1-ol.
ND: not determined

Avidekel oil extract were used in a prospective clinical study in patients with Crohn's disease described below. The investigational product contained 16.35% CBD, 4.01% THC (also Δ9-THC), 0.8% CBC, 0.74% CBG, 0.12% CBN and 0.08% CBDV, and terpenes, flavonoids, waxes and chlorophyll in certain proportions.

In terms of relative cannabinoid content, the investigational product contained THC:CBD ratio of 1:4.

In terms of total cannabinoid content, a drop of Avidekel oil estimated at approximately 0.04 ml contained approximately 1.6 mg THC and 6.54 mg CBD.

In terms of cannabinoid therapeutic dose, dosage, patients received 4-5 drops per administration, sublingual, 3-times per day. The control group received placebo containing olive oil and chlorophyll.

In the subsequently described retrospective study IBD patients were using Avidekel products with dose regimens as described above, alone or in combination with other THC enriched products administered by smoking or inhalation. A number of patients were using Avidekel products with products wherein THC:CBD are in equal proportions. Certain patients substituted Avidekel products with analogous CBD enriched products such as Refael administered by smoking or inhalation, according to personal preferences.

### Example 2

High CBD Compositions (Avidekel) are Effective for the Treatment of Crohn's Disease

Patients with differential diagnosis of Crohn's disease received Avidekel oil (N=18) or a placebo (N=21) administered sublingually as 4-5 drops, 3-times a day for a period of 8 weeks. All patients additionally received a classical anti-inflammatory therapy with at least one drug from group of immunosuppressants, e.g., azathioprine (Imuran), mercaptopurine (Purinethol), methotrexate (Rheumatrex), cyclosporine (Neoral); biological drugs, e.g. TNF inhibiting antibodies Adalimumab (Humira), Infliximab (Remicade); corticosteroids, e.g., prednisone (generic); anti-inflammatory 5-aminosalicylic acid (5-ASA), compounds (Delzicol, Asacol, Pentasa), or a combination thereof. Table 5 shows clinical characteristics of patients in the treatment and control groups.

TABLE 5
Clinical characteristics of patients at baseline.

| | Total | Cannabis | Placebo | P |
|---|---|---|---|---|
| | (n = 39) | (n = 18) | (n = 21) | value |
| Age | 35.1 ± 12.7 | 34.6 ± 14.3 | 35.6 ± 11.6 | NS |
| Gender male | 22 (56.4%) | 13 (72.2%) | 9 (42.9%) | 0.06 |
| Weight | 66.1 ± 18.6 | 66.6 ± 18.6 | 65.7 ± 19.0 | NS |
| CD-SES | 11.6 ± 5.6 | 10.8 ± 5.7 | 11.9 ± 5.1 | NS |
| DAI | 285.7 ± 94.4 | 279.3 ± 72.9 | 291.2 ± 111.1 | NS |
| QOL | 73.2 ± 16.9 | 76.0 ± 21.0 | 71.6 ± 13.7 | NS |
| WBC | 7.4 ± 2.5 | 7.1 ± 2.6 | 7.6 ± 2.5 | NS |
| HB, (g/dL) | 13.2 ± 1.6 | 13.6 ± 1.5 | 12.9 ± 1.6 | NS |
| HCT (%) | 40.3 ± 4.3 | 41.1 ± 4.4 | 39.6 ± 4.3 | NS |
| CRP | 2.4 ± 3.5 | 2.7 ± 4.7 | 2.1 ± 2.0 | NS |
| Calprotectin | 147.0 ± 104.5 | 153.6 ± 111.2 | 141.5 ± 102.1 | NS |

CD-SES: Simple Endoscopic Score for Crohn's Disease;
DAI: Crohn's Disease Activity Index;
QOL: Quality Of Life (SF-36);
WBS: White Blood cells count;
HB: Hemoglobin count;
HCT: Hematocrit count;
CRP: (7-reactive protein in blood;
Calprotectin: fecal;
NS: Non-Significant.

Avidekel oil used in the study comprised THC:CBD ratio of approximately 1:4 (w/w). In terms of dosage, Avidekel oil drop (0.04 ml volume) comprised approximately 3.7% THC and 15% CBD (w/w), and in terms of content—approximately 1.5 mg THC and 6 mg CBD.

In terms of an active drug dose per administration, an average drug dose was in the range of approximately 6-7.5 mg THC and 24-30 mg CBD. Doses did not exceed a maximum of 15 drops per administration (24 mg of THC and 98.1 mg of CBD).

In order to achieve an optimal therapeutic dose, each patient was subjected 3 weeks titration period, wherein the number of drops per administration and/or the number of administrations per day (morning, day, night) were gradually incremented. Optimal therapeutic dose was evaluated as a daily dose with a maximal impact on clinical indices of the disease with no significant adverse reactions.

In terms of daily doses, an average administration dose was in the range of approximately 18-23 mg THC and 72-90 mg CBD. Daily doses did not exceeded 400 mg actives, CBD and THC.

The result of this study, as reported by patients and physicians, showed significant improvements of clinical indices of Crohn's disease, general quality of life and treatment compliance in the group of patients treated with Avidekel oil compared to those in the placebo group. FIGS. 1A-1I show general trends observed in this study, in the Avidekel treated group (solid black lines) and placebo (dotted lines).

Specifically, the most significant beneficial effect were observed in CDAI and QOL (SF-36) scores. At 2- and 8-weeks' time points, CDAI score decreased from from 284.6±74.6 to 118.6±71.5 and QOL score increased from 74.0±19.8 to 96.3±17.6 in the Avidekel treated group compared to placebo wherein these scores remained relatively unchanged, i.e., CDAI of 286.7±112.0 to 212.6±102.4 and QOL of 72.6±13.8 to 79.9±16.2, respectively).

On the basis of this findings it is suggested that significant improvement in all study parameters could be observed in a study of a larger group of patients. Such study is currently ongoing. Comparative studies are currently carried out in patients with Crohn's disease, including oil extracts with THC:CBD ratio 1:6 and Sativex (ratio 1:1). A preliminary study including Avidekel oil (THC:CBD ratio 1:4) and a commercial preparation of dronabinol (Marinol, a synthetic THC in sesame oil) showed no significant effects of Marinol on clinical indices of the disease and general quality of life, and was therefore terminated due to relative side effects and poor patients' compliance.

Pharmacokinetic studies were performed in a group of patients from the above trial (N=7), while relating to two main cannabinoids, THC (Δ⁹-THC) and CBD, and two metabolites, 11-Hydroxy Δ⁹-THC (active metabolite) and Δ⁹ Carboxy THC (inactive metabolite). FIG. 2A-2D show mean cannabinoid blood levels after administration of a single dose of Avidekel oil (6.4 mg Δ⁹-THC and 26 mg CBD).

Specifically, maximal mean Δ⁹-THC values of 2.3±2.2 ng/mL were observed at 90 min until 120 min, with a continuous drop until 6 h. after administration wherein Δ⁹-THC levels were typical to those after 24 hours of Cannabis washout. A similar profile was observed for CBD pharmacokinetics with maximal mean CBD values of 6.2±5.9 ng/mL at 90 min until 120 min. with a continuous drop until 6 h. after administration. Regarding the two metabolites, 11-Hydroxy Δ⁹-THC maximal mean levels reached 4.5±4.2 ng/mL at 90 min until 120 min and dropped to 1.9±1.1 ng/mL. For Δ⁹ Carboxy THC in contrast, mean levels at after 90 min reached 34.5±47.7 ng/mL but continued to rise during the 6 h. period up to 75.5±77.0 ng/mL. These data need to be verified in further studies. Comparative pharmacokinetic studies are currently carried out in Crohn's patients, including oil extracts comprising THC:CBD ratio 1:6 and Sativex (ratio 1:1).

### Example 3

High THC Compositions (Erez) are Effective for the Treatment of Colitis

Patients with differential diagnosis of ulcerative colitis received Erez cigarettes (N=14) or placebo cigarettes (N=13), wherein Erez cigarettes (1 g. dry weight per cigarette, THC content of approximately 23% w/w and almost no CBD), or placebo cigarettes were administered at a daily dose of two cigarettes per day for a period of at least 8 weeks. All patients additionally received a classical anti-inflammatory therapy with at least one drug from group of immunosuppressants, biological drugs, corticosteroids, anti-inflammatory 5-ASA compounds or a combination thereof. Table 6 shows clinical characteristics of patients in the treatment and control groups.

TABLE 6
Clinical characteristics of patients at baseline.

| | Total | Cannabis | Placebo | P |
|---|---|---|---|---|
| | (n = 27) | (n = 14) | (n = 13) | value |
| Age | 33.5 ± 9.9 | 34.5 ± 11.5 | 32.6 ± 8.2 | NS |
| Gender male | 17 (63%) | 6 (42.9%) | 11 (84.6%) | <0.05 |
| Weight | 66.5 ± 15.7 | 71.3 ± 19.9 | 60.8 ± 5.5 | NS |
| MAYO | 2 (2-2) | 2 (2-2.5) | 2 (2-2) | NS |
| score (IQR) | | | | |
| DAI | 10.4 ± 3.9 | 10.2 ± 3.3 | 10.6 ± 2.8 | NS |
| QOL | 79.2 ± 12.9 | 79.2 ± 15.3 | 79.3 ± 10.6 | NS |
| WBC | 7.5 ± 2.8 | 6.6 ± 2.1 | 8.5 ± 3.2 | NS |
| HB | 13.5 ± 2.3 | 13.1 ± 2.7 | 13.9 ± 1.8 | NS |
| HCT | 41.0 ± 6.5 | 39.8 ± 7.6 | 42.2 ± 5.0 | NS |
| CRP | 1.2 ± 1.4 | 0.8 ± 0.9 | 1.6 ± 1.8 | NS |
| Calprotectin | 180.9 ± 117.2 | 135.4 ± 113.9 | 226.4 ± 109.3 | NS |

MAYO score: Mayo Scoring System for Assessment of Ulcerative Colitis Activity;
IQR: Interquartile Range;
DAI: Disease Activity Index;
QOL: Quality Of Life (SF-36);
WBS: White Blood cells count;
HB: Hemoglobin count;
HCT: Hematocrit count;
CRP: C-reactive protein in blood;
Calprotectin: fecal;
NS: Non-Significant.

Erez cigarettes used in the study comprised 23% THC (w/w) with almost no traceable CBD, and in terms of content—approximately 0.23 gr. THC.

In terms of an active drug dose per administration, since Erez was consumed by smoking or inhalation, this makes estimation of the administered drug dose or a daily dose highly inaccurate and highly dependent on personal use. Daily doses did not exceed a maximum of 2 cigarettes per day.

The result of this study, as reported by patients and physicians, showed significant improvements of clinical indices of colitis, general quality of life and treatment compliance in the group of patients treated with Erez compared to placebo. FIGS. 3A-31 show general trends observed in this study, the Avidekel treated group (solid black lines) and placebo (dotted lines).

Specifically, the most significant beneficial effect were observed in DAI and QOL (SF-36).scores. In the Erez group DAI score decreased from 10.2±3.3 at the baseline to 3.9±3.3 after 8 weeks treatment compared to 10.6±2.8 to 8.2±2.1 in the placebo (p<0.01). Analogously, QOL score in the Erez group increased from 76.0±21.0 to 99.6±19.2 comparrent to 71.6±13.7 to 80.8±14.0 in placebo (p<0.01).

On the basis of this findings it is suggested that significant improvement in all study parameters could be observed in a study of a larger group of patients. Such study is currently ongoing.

Further comparative studies are currently conducted in patients with various types of IBD treated with compositions of the invention with various THC:CBD:terpene content (ratio) as opposed to other commercially available cannabinoid compositions.

### Example 4

Avidekel Oil—Extract of is Effective for Prolonged Management and Treatment of IBD

Patients diagnosed with IBD (N=50) received an oil-based extract of Avidekel or a placebo oil, the preparations were orally administered as 4-5 drops, three times a day, for a period of 8 weeks. Drug regiments were similar to EXAMPLE 2 as well monitoring of the disease condition.

After monitoring for at least 8 weeks, patients and physicians reported significant improvements relating to all clinical indices of IBD and also to indices of general quality of life and treatment compliance. Patients were compliant with Avidekel oil being administered during waking hours.

### Example 5

Erez Cigarettes are Effective for an Immediate Alleviation of IBD Symptoms

Patients diagnosed with IBD (N=30) received Erez cigarettes (0.5-1 g. dry weight per cigarette) consumed as two cigarettes per day for a period of at least 8 weeks, or placebo cigarettes. Drug regiments were similar to EXAMPLE 3 as well monitoring of the disease condition.

During the monitoring period and two weeks after, patients and physicians reported significant improvements relating to immediate relief of symptoms of IBD, including pain. Patients were more compliant with administration of Erez cigarettes before sleep.

### Example 6

Midnight is an Effective Substitute for Erez in Non-Compliant Patients

IBD patients non-compliant with Erez due to psychotropic effects (about 20%). received Midnight in the form of cigarettes wherein THC:CBD content is approximately equal, administered as two cigarettes per day for a period of at least 8 weeks, during the day and/or before sleep. Midnight proved to be an effective substitute for Erez, particularly considering patients and physicians reporting on immediate relief of IBD symptoms, including pain, gain of appetite, and improvement in general quality of life, in absence of or with a significant reduction of adverse events compared to a previous experience with Erez.

### Example 7

Surprising Beneficial Effects of Combination Therapies for a Long Term Treatment and Management of IBD

Data on patients with clinical diagnosis of IBD, Crohn's disease and colitis, was selected for this study. Data included clinical anamnesis, physical examination and clinical evaluation of IBD in relating to DAI clinical severity score, biochemical and blood tests for indices of inflammation, calprotectin, and colonoscopy using standard scores, and also evaluation of patient's life-style, quality of life, personal preferences retrieved from QOL (SF-36) questionnaires. Additional data included reporting of adverse events during the first month and the first year.

The IBD group (N=291) included 169 males (58%), mean age 39.8 years (SD=16.9) with 142 patients having more than 6 months experience with phyto-derived compositions of the invention (49%). The majority of patients reported on pain (94%) of various degrees on a subjective severity scale (0-10). Within the group of 142 patients completing the follow-up questionnaire, 65 patients (46%) reported on preference to a combination therapy including CBD enriched and THC enriched compositions administered in succession, CBD enriched composition—preferably during the day, and THC enriched compositions—preferably before sleep; 77 patients (54%) reported preference of a monotherapy—preferably THC enriched compositions; 8 patients (6%) reported on preference to Midnight including approximately equal THC:CBD content.

Data analysis of patients receiving a combination therapy versus those receiving a monotherapy showed significant improvement of disease indices in the combination therapy group compared to monotherapy group (p<0.001). Most notably, patients receiving combination therapy with CBD-enriched and THC enriched compositions (in succession) performed better than patients treated with compositions comprising THC and CBD in equal amounts (Midnight).

Further patients receiving combination therapy reported on significantly lower number and severity of adverse events (nausea, dizziness, dry eyes syndrome, psychoactive symptoms, sleepiness, general weakness) compared to the monotherapy group (p<0.001). Also in this analysis, patients treated with CBD-enriched and THC enriched combination therapy in succession performed better than patients treated with Midnight (THC:CBD equal).

Further, in the analysis of a reduction of pain, patients on CBD-enriched and THC enriched combination therapy reported on a more significant alleviation of pain, in terms of number of incident, severity score and longitudinal management of pain, than patients on monotherapy (p<0.001), including Midnight. This finding is moreover surprising since THC-enriched strains and cannabinoid compositions have been considered, so far, more effective for the treatment of pain.

These finding suggest that combination therapies including CBD-enriched and THC enriched compositions of the invention are more efficacious for long term treatment and management of IBD than monotherapies, even those including THC and CBD. These effects can be further enhanced by specific dose regiments and personalized approached. Studies of advantageous effects of combination therapies using various phyto-derived material, methods of extraction and dose regimens are currently ongoing.

### Specific Embodiments of the Invention

In one of its aspects the invention relates to compositions comprising a pre-defined ratio of tetrahydrocannabinol (THC):cannabidiol (CBD) for use in a method for treating, alleviating or reducing at least one symptom of a condition related to Inflammatory Bowel Disease (IBD), the composition optionally further comprising at least one of a carrier, a buffer, an excipient.

In numerous embodiments compositions of the invention are derived from a dry resin-producing pistillate inflorescences of a female Cannabis plant (Cannabis flowers) or an extract thereof, said resin or extract comprising in the a pre-defined ratio of THC:CBD.

In other embodiments the compositions can comprise at least one of THC, CBD is a synthetic, semi-synthetic or purified from a Cannabis plant.

In specific embodiments the compositions can comprise a ratio of THC:CBD of at least about 1:1 per weight (w/w), or substantially close to 1:1.

In yet other embodiments the compositions of the invention are enriched in THC or CBD, or comprising a ratio of THC:CBD other than 1:1 (w/w).

In specific embodiments the compositions can comprise a ratio of THC:CBD in a range between at least about 1.5:1-2:1, 2:1-3:1, 3:1-5:1, 5:1-10:1, 10:1-50:1, 50:1-100:1, 100:1-500:1, 100:1-1000:1 (w/w), respectively, or more.

In further embodiments the compositions can comprise substantially no CBD.

In numerous embodiments the compositions of the invention can comprise a ratio of THC:CBD in a range between at least about 1:1.5-1:2, 1:2-1:3, 1:3-1:4, 1:4-1:5, 1:5-1:10, 1:10-1:20, 1:20-1:30, 1:30-1:40, 1:40-1:50, 1:50-1:100, 1:100-1:500, 1:500-1:1000 (w/w), respectively, or less.

In further embodiments the compositions can comprise substantially only CBD.

In specific embodiments the compositions of the invention are in a dosage form of a cigarette comprising a phyto-derived material comprising a THC content in a range between at least about 10-30%, 12-28%, 13-27%, 14-26%, 15-25%, 16-24%, 17-23%, 18-22%, or approximately 20% (w/w).

In further embodiments such compositions comprise a material derived from a C. Indica strain designated as ‘Erez’.

In specific embodiments the compositions of the invention are in an oral dosage form of a phyto-derived oil extract of comprising a CBD content in a range between at least about 10-30%, 10-20%, 11-19%, 12-18%, 12.5-17.5%, 13-17%, 13.5-16.5%, 14-16%, 14.5-15.5%, or approximately 15% (w/w).

In further embodiments said oil extract can further comprise a THC content in the range between at least about 0.1-7.5%, 0.5-7%, 0.5-6%, 0.5-5%, 0.5-4%, 0.5-3%, 0.5-2%, or 0.5-1% (w/w).

In still further embodiments such compositions comprise a material derived from a C. Indica strain designated as ‘Avidekel’.

In numerous embodiments the compositions of the invention are in a dosage form of a cigarette comprising a phyto-derived material comprising a substantially equal THC and CBD content in a range between at least about 5-30%, 5-20%, 6-19%, 7-18%, 8-17%, 9-16%, 10-15%, 10-14%, 10-13%, 10-12%, or 10-11% (w/w).

In specific embodiment said compositions comprise a material derived from a C. Sativa L. strain designated as ‘Midnight’.

In specific embodiments the compositions of the invention are adapted for inhalation and/or vaporization.

In numerous embodiments the compositions of the invention are intended for use in a method for treating alleviating or reducing at least one symptom of a condition related to IBD, said alleviating or reducing of a symptom being immediate.

In yet other embodiments the compositions are intended for use in a method for treating, alleviating or reducing at least one symptom of a condition related to IBD, said alleviating or reducing of a symptom being prolonged.

In specific embodiments the compositions are intended for use in a method for treating alleviating or reducing at least one symptom of ulcerative colitis.

In yet other embodiments the compositions are intended for use in a method for treating alleviating or reducing at least one symptom of Crohn's disease.

It is yet another aspect of the invention to provide Cannabis-based oral compositions enriched in CBD for use in a method for a prolonged treatment, alleviation or a reduction of at least one symptom of a condition related to IBD.

In specific embodiments such compositions are applicable to a prolonged treatment, alleviation or a reduction of at least one symptom of Crohn's disease.

In yet another aspect the invention provides Cannabis-based compositions enriched in THC for use in a method for an immediate treatment, alleviation or a reduction of at least one symptom of a condition related to IBD.

In specific embodiments the compositions as above are adapted for at least one of smoking, inhalation, vaporization.

In certain embodiments such compositions are applicable to an immediate treatment, alleviation or a reduction of is ulcerative colitis.

In numerous embodiments the compositions of the invention can further comprise at least one additional therapeutic agent.

In specific embodiments the therapeutic agent is at least one of an anti-inflammatory, an anti-nociceptive, an antibiotic, an antiemetic, an anti-diarrheal drug.

Its yet another aspect of the invention to provide methods for treating, alleviating or reducing at least one symptom of a condition related to IBD in a subject in need thereof, said methods comprise administering to said subject a therapeutically effective amount of at least one composition comprising a pre-defined ratio of THC:CBD.

In numerous embodiments said treating, alleviating or reducing of a symptom is immediate and/or prolonged.

In specific embodiments the compositions administered in said methods comprise at least one of THC, CBD is a synthetic, semi-synthetic or purified from a Cannabis plant, or in a form of a Cannabis plant derived material (a Cannabis flower derived material) or an extract thereof, or any combination thereof.

In certain embodiments the methods of the invention can comprise administering to the subject more than one composition, each composition comprising a distinct pre-defined ratio of THC:CBD, the administering is consecutive.

In numerous embodiments the methods of the invention comprise administering to the subject at least one of

(i) a composition comprising a ratio of THC:CBD of at least about 1:1 w/w, or substantially close to 1:1,

(ii) a composition enriched in THC or comprising substantially no CBD,

(iii) a composition enriched in CBD or comprising substantially only CBD, or a consecutive administering of a combination thereof.

In yet other embodiments, the methods can comprise administering to the subject at least one of

(i) at least one cigarette comprising a phyto-derived material comprising a THC content in a range between at least about 10-30%, 12-28%, 13-27%, 14-26%, 15-25%, 16-24%, 17-23%, 18-22%, or about 20% (w/w),

(ii) at least one oral dosage form of a phyto-derived oil extract of comprising a CBD content in a range between at least about 10-30%, 10-20%, 11-19%, 12-18%, 12.5-17.5%, 13-17%, 13.5-16.5%, 14-16%, 14.5-15.5%, or about 15% (w/w), and further optionally comprising a THC content in a range between at least about 0.1-7.5%, 0.5-7%, 0.5-6%, 0.5-5%, 0.5-4%, 0.5-3%, 0.5-2%, 0.5-1% (w/w).

(iii) at least one cigarette comprising a phyto-derived material comprising an substantially equal content of THC and CBD, in a range between at least about 5-30%, 5-20%, 6-19%, 7-18%, 8-17%, 9-16%, 10-15,% 10-14%, 10-13%, 10-12%, 10-11% (w/w). or a consecutive administering of a combination thereof.

In specific embodiments the methods of the invention comprise administering to the subject at least one of

(i) at least one cigarette comprising a phyto-derived material of Erez,

(ii) at least one oral dosage form of a phyto-derived oil extract of Avidekel,

(iii) at least one cigarette comprising phyto-derived material of Midnight, or a consecutive administering of a combination thereof.

In yet further embodiments in the above methods the Erez and/or Midnight derived materials are in a form adapted for inhalation and/or vaporization.

In numerous embodiments the methods of the invention can further comprise consecutive or simultaneous administering of at least one additional therapeutic agent.

In specific embodiments the additional therapeutic agent is at least one of an anti-inflammatory, an anti-nociceptive, an antibiotic, an antiemetic, an anti-diarrheal drug.

In yet another aspect the invention provides methods for an immediate treatment, alleviation or reduction at least one symptom of a condition related to IBD in a subject in need thereof, such methods comprise administering to the subject at least one of

(i) at least one cigarette comprising a phyto-derived material comprising a THC content in a range between at least about 10-30%, 12-28%, 13-27%, 14-26%, 15-25%, 16-24%, 17-23%, 18-22%, or about 20% (w/w),

(ii) at least one cigarette comprising a phyto-derived material comprising a substantially equal content of THC and CBD in a range between at least about 5-30%, 5-20%, 6-19%, 7-18%, 8-17%, 9-16%, 10-15,% 10-14%, 10-13%, 10-12%, 10-11% (w/w).

In yet another aspect the invention provides methods for a prolonged treatment, alleviation or reduction of at least one symptom of a condition related to IBD in a subject in need thereof, such methods comprise administering to the subject at least one oral dosage form of an oil extract of a phyto-derived material comprising a CBD content in a range between at least about 10-30%, 10-20%, 11-19%, 12-18%, 12.5-17.5%, 13-17%, 13.5-16.5%, 14-16%, 14.5-15.5%, or about 15% (w/w), and further optionally comprising a THC content in a range between at least about 0.1-7.5%, 0.5-7%, 0.5-6%, 0.5-5%, 0.5-4%, 0.5-3%, 0.5-2%, 0.5-1% (w/w).

In specific embodiments the above methods comprise administering to the subject at least one of

(i) at least one cigarette comprising a phyto-derived material of Erez,

(ii) at least one cigarette comprising a phyto-derived material of Midnight.

In further embodiments the methods according to the above can comprise further administering to the subject at least one oral dosage form of an oil extract of phyto-derived material of Avidekel.

In yet another aspect the invention provides methods for treating, alleviating or reducing at least one symptom of Crohn's disease in a subject in need thereof, such methods comprise administering to the subject at least one oral dosage form of an oil extract of phyto-derived material comprising a CBD content in a range between at least about 10-30%, 10-20%, 11-19%, 12-18%, 12.5-17.5%, 13-17%, 13.5-16.5%, 14-16%, 14.5-15.5%, about 15% (w/w), and further optionally comprise a THC content in a range between at least about 0.1-7.5%, 0.5-7%, 0.5-6%, 0.5-5%, 0.5-4%, 0.5-3%, 0.5-2%, 0.5-1% (w/w).

In specific embodiments the methods according to the above comprise administering to the subject at least one oral dosage form of an oil extract of a phyto-derived material of Avidekel.

In yet another aspect the invention provides methods for treating, alleviating or reducing at least one symptom of ulcerative colitis in a subject in need thereof, such methods comprise administering to the subject at least one cigarette comprising a phyto-derived material comprising a THC content in a range between at least about 10-30%, 12-28%, 13-27%, 14-26%, 15-25%, 16-24%, 17-23%, 18-22%, or about 20% (w/w).

In specific embodiments the methods according to the above comprise administering to the subject at least one cigarette comprising a phyto-derived material of Erez.

It is another aspect of the invention to provide use of a composition for the manufacture/preparation of a medicament for treating, alleviating or reducing at least one symptom of a condition related to IBD, the composition comprising a pre-defined ratio of THC and CBD, and further optionally further comprising at least one of a carrier, a buffer, an excipient.

Annex A.
Terpene analysis of Cannabis strains of the invention
Cannabis strain Omer (30% sativa, 70% indica)
Terpenes:

| 9.415 | 100.000% | 7.43 | myrcene |
|---|---|---|---|
| 17.890 | 69.569% | 25.36 | β-caryophyllene |
| 8.058 | 51.859% | 5.85 | α-pinene |
| 21.489 | 45.834% | 34.79 | β-euclesinol |
| 19.818 | 43.843% | 30.66 | selinα-3,7(11)-diene |
| 21.035 | 33.048% | 31.77 | 10-epi-γ-eudesmol |
| 20.600 | 31.970% | 32.92 | guaiol |
| 18.468 | 31.023% | 26.82 | α-humulene |
| 21.837 | 26.675% | 36.17 | α-bisabolol |
| 18.285 | 26.601% | 26.92 | trans-β-farnesene |
| 21.605 | 25.735% | 35.60 | bulnesol |
| 9.145 | 16.775% | 7.04 | β-pinene |
| 10.347 | 13.128% | 8.69 | limonene |
| 11.863 | 11.546% | 11.32 | linalool |
| 13.697 | 10.799% | 15.40 | hexyl butanoate |
| 19.461 | 10.414% | 29.70 | β-sesquiphellandrene |
| 19.200 | 9.967% | 29.04 | β-bisabolene |
| 18.998 | 7.086% | 28.37 | β-selinene |
| 19.972 | 7.059% | 30.09 | cis-nerolidol |
| 13.822 | 5.627% | 15.21 | α-terpineol |
| 20.436 | 5.108% | 32.16 | caryophyllene oxide |
| 12.315 | 4.172% | 12.28 | exo-fenchol |
| 17.706 | 3.466% | 25.10 | α-cis-bergamotene |
| 10.712 | 3.453% | 9.42 | β-ocimene |
| 12.459 | 3.220% | trans-pinene hydrate | |
| 13.399 | 2.963% | 14.29 | borneol |
| 5.520 | 2.700% | ethyl-cyclohexane | |
| 11.960 | 2.575% | 11.51 | nonanal |
| 20.764 | 2.144% | 33.17 | 5-ept-7-epi-α-eudesmol |
| 5.424 | 1.988% | 1.2-dimethyl-cis-cyclohexane | |
| 17.822 | 1.333% | 25.31 | α-santalene |
| 7.203 | 1.272% | 4.98 | heptanal |
| 8.462 | 1.138% | 6.26 | camphene |
| 17.014 | 0.994% | 23.43 | ylangene |
| 12.824 | 0.703% | hexyl-isobutyrate | |
| 6.741 | 0.572% | 1,2,4-trimethyl-cyclohexane | |
| 11.671 | 0.546% | 10.88 | fenchone |
| 17.264 | 0.485% | 24.84 | sesquithajene |

| | | |
|---|---|---|
| 7.414 | 0.411% | cis-1-ethy-3-methylcyclohexane |

Cannabis strain Avidekel (60% indica, 40% sativa)
Terpenes:

| | |
|---|---|
| 100.000% | myrcene |
| 55.892% | α-eudesmol |
| 45.532% | guaiol |
| 45.318% | 10-epi-γ-eudesmol |
| 42.212% | bulnesol |
| 39.710% | α-pinene |
| 37.571% | β-caryophyllene |
| 25.614% | epi-α-bisabolol |
| 21.321% | β-pinene |
| 17.376% | limonene |
| 13.427% | α-humulene |
| 13.044% | cis-β-farnesene |
| 9.225% | trans-α-bergamotene |
| 9.222% | γ-eudesmol |
| 8.872% | endo-fenchol |
| 7.877% | linalool |
| 7.415% | cis-α-bisabolene |
| 6.943% | trans-pinene hydrate |
| 6.037% | α-terpineol |
| 5.838% | β-eudesmol |
| 5.426% | β-bisabolene |
| 4.461% | borneol |
| 4.020% | caryophyllene oxide |
| 3.779% | cis-nerolidol |
| 3.577% | 5-epi-7-epi-α-eudesmol |
| 3.071% | trans, trans-α-farnesene |
| 2.838% | nonanal |
| 2.745% | β-sesquiphellandrene |
| 2.695% | valencene |
| 2.020% | ipsdienol |
| 2.017% | selina-3,7(11)-diene |
| 1.846% | humulene epoxide II |
| 1.523% | cis-pinene hydrate |
| 1.495% | 1,8-cineole |
| 1.425% | cis-α-bergamotene |
| 1.308% | camphene |
| 1.136% | heptanal |
| 0.933% | fenchone |
| 0.802% | γ-curcumene |
| 0.643% | β-eurcumene |

Cannabis strain Barak (70% indica, 30% sativa
Terpenes:

| | |
|---|---|
| 100.000% | myrcene |
| 54.246% | β-caryophyllene |
| 32.085% | germacrene B |
| 29.293% | α-pinene |
| 29.168% | γ-elemene |
| 28.639% | β-endesmol |
| 27.663% | selina-3,7(11)-diene |
| 27.222% | 10-epi-γ-endesmol |
| 24.857% | guaiol |
| 73.254% | bulnesol |
| 22.796% | α-humulene |
| 19.084% | trans-β-farnesene |
| 16.913% | β-pinene |
| 16.624% | limonene |
| 11.927% | α-bisabolol |
| 10.203% | linalool |
| 9.834% | trans-α-bergamotene |
| 6.277% | trans, trans-α-farnecsene |
| 5.673% | β-bisabolene |
| 4.544% | β-selinene |
| 4.165% | α-selinene |
| 3.919% | exo-fenchol |
| 3.509% | juniper camphor |
| | (=eudcsm-7(11)-en-4-ol) |
| 3.101% | trans-pinene hydrate |
| 2.717% | trans-β-ocimene |
| 2.537% | α-eudesmol 5.676% α-terpineol |
| 1.964% | hexyl hexanoate |
| 1.958% | β-phellandrene |
| 1.874% | cis-α-bergamotene |
| 1.695% | borneol |
| 1.320% | ipsdienol |
| 1.207% | heptanal |
| 1.166% | cis-pinene hydrate |
| 1.128% | amphene |
| 1.113% | p-xylene |
| 0.925% | fenchone |

Cannabis strain Erez (70% indica, 30% saliva)
Terpenes:

| | |
|---|---|
| 100.000% | myrcene |
| 83.674% | β-caryophyllene |
| 65.643% | selinα-3,7(11)-diene |
| 55.953% | γ-selinene |
| 47.281% | 10-epi-γ-eudesmol |
| 46.373% | β-eudesmol |
| 41.174% | guaiol |
| 35.863% | α-humulene |
| 33.921% | bulnesol |
| 29.604% | α-bisabolol |
| 27.110% | α-pinene |
| 21.362% | germacrene B |
| 17.679% | γ-elemene |
| 16.263% | trans-α-bergamotene |
| 15.793% | β-pinene |
| 15.429% | limonene |
| 15.375% | linalool |
| 7.988% | β-bisabolene |
| 6.508% | α-terpineol |
| 6.481% | pentadecanol |
| 6.287% | caryophyllene oxide |
| 6.164% | eudesm-7,11-en-4-ol |
| | (=juniper camphor) |
| 5.430% | hinesol |
| 5.004% | α-eudesmol |
| 4.951% | endo-fenchol |
| 4.649% | trans-nerolidol |
| 4.351% | β-selinene |
| 4.168% | trans-pinene hydrate |
| 4.146% | trans, trans-α-farnesene |
| 3.512% | trans-β-ocimene |
| 3.154% | α-selinene |
| 2.238% | borneol |
| 2.104% | humulene epoxide II |
| 2.093% | 5-epi-7-ept-α-eudesmol |
| 1.700% | hexyl hexanoate |
| 1.505% | heptanal |
| 1.419% | β-phellandrene |
| 1.352% | ipsdienol |
| 1.331% | fenchone |
| 1.076% | camphene |

Cannabis strain Jasmin (70% indica, 30% sativa)
Terpenes:

| | |
|---|---|
| 100.000% | myrcene |
| 44.457% | α-pinene |
| 26.344% | β-pinene |
| 20.411% | limonene |
| 16.273% | germacrene B |
| 15.405% | β-caryophyllene |
| 15.161% | β-eudesmol |
| 14.834% | selina-3,7(11)-diene |
| 14.627% | 10-epi-γ-eudesmol |
| 13.566% | guaiol |
| 11.538% | bulnesol |
| 7.130% | α-bisabolol |
| 5.634% | α-humulene |
| 5.480% | γ-elemene |
| 5.073% | trans, trans-α-farnesene |
| 4.911% | exo-fenchol |
| 4.844% | linalool |
| 4.092% | α-terpineol |
| 3.854% | trans-pinene hydrate |
| | (=trans-2-pinanol) |
| 1.611% | citronellol |
| 1.534% | cis-β-farnesene |
| 1.519% | borneol |
| 1.479% | β-selinene |
| 1.464% | juniper camphor |
| | =eudesm-7(11)-en-4-ol) |
| 1.422% | camphene |
| 1.275% | ipsdienol |
| 1.265% | β-bisabolene |
| 1.180% | α-selinene |
| 1.151% | fenchone |
| 1.106% | nonanal |
| 0.795% | heptanal |
| 0.695% | trans-α-bergamotene |
| 0.598% | cis-pinene hydrate |
| | (=cis-2-pinanol) |

Cannabis strain Tal (70% indica, 30% sativa)
Terpenes:

| | |
|---|---|
| 100.000% | myrcene |
| 50.610% | trans-β-caryophyllene |
| 28.076% | α-pinene |
| 26.822% | γ-elemene |
| 26.160% | 30.66 selina-3,7(11)-diene |
| 25.276% | germacrene B |
| 24.585% | 10-epi-γ-eudesmol |
| 23.903% | β-eudesmol |
| 21.819% | bulnesol |
| 21.224% | guaiol |
| 20.675% | α-humulene |
| 19.317% | trans-β-farnesene |
| 18.042% | limonene |
| 14.962% | β-pinene |
| 14.466% | α-bisabolol |
| 9.464% | trans-α-bergamotene |
| 8.032% | linalool |
| 5.006% | β-sesquiphellandrene |
| 4.885% | β-bisabolene |
| 4.805% | α-terpineol |
| 3.176% | trans-pinene hydrate |
| 2.958% | endo-fenchol |
| 2.917% | β-selinene |
| 2.840% | trans-β-ocimene |
| 2.807% | eudesm-7(11)-en-4-ol |
| | (=juniper camphor) |
| 2.791% | α-eudesmol |
| 2.473% | trans, trans-α-farnesene |
| 2.100% | β-eudesmol |
| 1.994% | cis-α-bergamotene |
| 1.744% | α-selinene |
| 1.593% | borneol |
| 1.488% | hexyl hexanoate |
| 1.157% | 5-epi-7-epi-α-eudesmol |
| 0.953% | camphene |
| 0.903% | cis-pinene hydrate |
| 0.697% | fenchone |
| 0.441% | heptanal |

Cannabis strain Shira 70% sativa, 30% indica)
Terpenes:

| | |
|---|---|
| 100.000% | myrcene |
| 99.732% | γ-elemene |
| 80.539% | β-caryophyllene |
| 39.173% | selina-3,7(11)-diene |
| 39.025% | α-pinene |
| 32.667% | limonene |
| 29.731% | β-pinene |
| 24.434% | α-humulene |
| 22.157% | germacrene B |
| 13.871% | exo-fenchol |
| 12.627% | α-terpineol |
| 12.554% | trans-pinene hydrate |
| 9.904% | α-guiaiene |
| 8.126% | α-selinene |
| 7.508% | β-selinene |
| 7.252% | β-eudesmol |
| 4.897% | linatool |
| 4.467% | β-bisabolene |
| 4.100% | bornenol |
| 3.872% | ipsdienol |
| 3.654% | caryophyllene oxide |
| 3.393% | cis-pinene hydrate |
| 3.032% | n-hexadecanol |
| 2.880% | trans-β-farnesene |
| 2.409% | hexyl hexanoate |
| 2.326% | camphene |
| 2.178% | nonanal |
| 1.976% | fenchone |
| 1.118% | heptanal |

Cannabis strain Or (70% indica, 30% sativa)
Terpenes:

| | |
|---|---|
| 100.000% | α-pinene |
| 84.363% | β-caryophyllene |
| 65.284% | γ-elemene |
| 52.205% | β-pinene |
| 41.511% | germacrene B |
| 37.867% | β-eudesmol |
| 35.642% | myrcene |
| 35.411% | α-humulene |
| 34.099% | selina-3,7(11)-diene |
| 28.043% | guaiol |
| 27.663% | 10-epi-γ-endesmol |
| 25.285% | bulnesol |
| 23.736% | cis-β-farnesene |
| 22.100% | α-bisabolol |
| 15.336% | trans-α-bergamotene |
| 12.405% | α-guaiene |
| 9.237% | linalool |
| 7.791% | α-selinene |
| 7.543% | exo-fenchol |
| 6.693% | α-terpineol |
| 6.491% | β-selinene |
| 5.479% | trans-pinene hydrate |
| 4.946% | caryophyllene oxide |
| 4.536% | hinesol |
| 3.806% | β-bisabolene |
| 7.853% | cis-α-bergamotene |
| 2.392% | eudesm-7(11)-n-4-ol |
| | (juniper camphor) |
| 2.305% | borneol |
| 2.295% | β-sesquiphellandrene |
| 2.157% | camphene |
| 2.062% | limonene |
| 1.964% | 5-epi-7-epi-α-eudesmol |
| 1.950% | citronellol |
| 1.867% | heptanal |
| 1.750% | humulene epoxide II |
| 1.478% | methyl hexadecanoate |
| 1.476% | nonanal |
| 1.410% | cis-pinene hydrate |
| 1.323% | 1,8-cineole |
| 0.582% | cis-sabinene hydrate |

Cannabis strain Mango (70% sativa, 30% indica)
Terpenes:

| | |
|---|---|
| 100.000% | β-caryophyllene |
| 67.704% | myrcene |
| 41.365% | α-humulene |
| 34.607% | linalool |
| 30.731% | cis-β-farnesene |
| 29.919% | β-eudesmol |
| 25.576% | bulnesol |
| 25.270% | guaiol |
| 23.394% | 10-epi-γ-eudesmol |
| 16.760 | 24.84 sesquithujene (28.53 |
| | (α-zingiberene) 1.091% |
| 13.262% | β-pinene |
| 9.314% | endo-fenchol |
| 8.492% | cis-nerolidol (nerolidol) |
| 7.743% | α-pinene |
| 7.583% | α-terpineol |
| 7.217% | trans-pinene hydrate |
| | (=cis-2-pinanol) |
| 4.576% | β-sesquiphellandrene |
| 4.454% | β-bisabolene |
| 4.438% | trans-α-bergamotene |
| 4.054% | hinesol |
| 3.393% | α-bisabolol |
| 3.335% | α-eudesmol |
| 3.034% | caryophyllene oxide |
| 2.407% | borneol |
| 1.850% | camphene |
| 1.517% | 5-epi-7-epi-α-eudesmol |
| 1.314% | cis-pinene hydrate |
| | (=cis-2-pinanol) |
| 1.143% | nonanal |

Cannabis strain Refael (80% sativa, 20% indica)
Terpenes:

| | |
|---|---|
| 100.000% | β-caryophyllene |
| 81.299% | epi-α-bisabolol |
| 66.640% | myrcene |
| 58.001% | β-eudesmol |
| 53.445% | 10-epi-γ-endesmol |
| 50.596% | guaiol |
| 38.938% | bulnesol |
| 35.776% | α-humulene |
| 32.730% | linalool |
| 22.619% | limonene |
| 14.375% | fenchol |
| 13.102% | α-terpineol |
| 11.567% | β-bisabolene |
| 11.050% | trans-pinene hydrate |
| 8.853% | β-pinene |
| 8.751% | caryophyllene oxide |
| 5.996% | cis-β-farnesene |
| 5.459% | selina-3,7(11)-diene |
| 5.161% | trans-nerolidol |
| 4.823% | borneol |
| 4.731% | α-pinene |
| 3.587% | β-selinene |
| 3.453% | eudesm-7(11)-en-4-ol |
| | (=juniper camphor) |
| 3.203% | valencene |
| 3.026% | 5-epi-7-epi-α-eudesmol |
| 2.994% | trans-α-bergamotene |
| 2.123% | cis-pinene hydrate |
| 1.943% | humulene epoxide II |
| 1.737% | α-selinene |
| 1.439% | fenchone |
| 1.048% | camphene |
| 0.589% | heptanal |

Cannabis strain El-na (60% indica, 40% sativa)
Terpenes:

| | |
|---|---|
| 100.000% | α-eudesmol |
| 96.130% | β-caryophyllene |
| 93.110% | myrcene |
| 86.289% | guaiol |
| 86.091% | 10-epi-γ-endesmol |
| 76.042% | epi-α-bisabolol |
| 73.352% | α-pinene |
| 70.917% | bulnesol |
| 42.086% | β-pinene |
| 35.083% | cis-α-bisabolene |
| 30.717% | α-humulene |
| 26.115% | limonene |
| 24.873% | selina-3,7(11)-diene |
| 16.754% | endo-fenchol (fenchyl alcohol) |
| 15.032% | γ-eudesinol |
| 13.364% | trans-pinene hydrate |
| 12.565% | γ-elemene |
| 10.596% | linalool |
| 9.852% | germacrene B |
| 9.314% | trans-β-farnesene |
| 7.487% | borneol |
| 7.284% | β-bisabolene |
| 6.790% | caryophyllene oxide |
| 5.296% | cis-linalool oxide |
| 5.290% | 5-epi-7-epi-α-eudesmol |
| 4.797% | trans-nerolidol |
| 3.977% | nonanal |
| 3.423% | α-terpineol |
| 3.258% | ipsdienol |
| 3.144% | cis-pinene hydrate |
| 3.085% | hexyl hexanoate |
| 2.863% | decanal |
| 2.745% | trans-α-bergamotene |
| 2.730% | camphene |
| 1.173% | heptanal |
| 1.128% | fenchone |

Cannabis strain Alaska (70% sativa, 30% indica)
Terpenes:

| | |
|---|---|
| 100.000% | β-eudesmol |
| 85.877% | guaiol |
| 82.285% | bulnesol |
| 81.350% | 10-epi-γ-eudesmol |
| 73.393% | α-bisabolol |
| 66.753% | α-pinene |
| 65.412% | trans-caryophyllene |
| 50.195% | myrcene |
| 45.857% | limonene |
| 45.707% | β-pinene |
| 33.839% | linalool |
| 48.390% | γ-elemene |
| 43.351% | cis-α-bisabolene |
| 36.917% | germacrene B |
| 30.334% | selina-3,7(11)-diene |
| 24.540% | exo-fenchyl alcohol |
| 21.954% | α-terpineol |
| 19.318% | α-humulene |
| 15.563% | γ-eudesmol |
| 15.225% | trans-pinene hydrate |
| 14.730% | trans-β-ocimene |
| 14.209% | β-bisabolene |
| 10.476% | eudesmol |
| 6.314% | cis-β-farnesene |
| 5.968% | borneol |
| 5.860% | trans, trans-α-farnesene |
| 5.830% | 5-epi-7-epi-α-eudesmol |
| 3.487% | valencene |
| 3.152% | camphene |
| 2.815% | β-selinene |
| 1.738% | heptanal |
| 1.676% | α-selinene |
| 1.468% | nonanal |
| 1.319% | hinesol |
| 0.7/5% | cis-sabinene hydrate |
| 0.641% | camphene hydrate |
| 0.544% | fenchone |

Cannabis strain Eran Almog (80% indica, 20% sativa)
Terpenes:

| | |
|---|---|
| 100.000% | β-caryophyllene |
| 88.456% | β-eudesmol |
| 68.327% | myrcene |
| 66.011% | guaiol |
| 64.969% | 10-epi-7-eudesmol |
| 64.318% | α-pinene |
| 56.425% | bulnesol |
| 47.732% | selina-3,7(11)-diene |
| 41.806% | limonene |
| 39.171% | α-bisabolol |
| 33.801% | α-humulene |
| 32.039% | β-pinene |
| 31.903% | trans-β-farnesene |
| 24.499% | trans-α-bergamotene |
| 17.578% | linalool |
| 14.810% | exo-fenchol |
| 14.169% | γ-elemene |
| 14.676% | α-bulnesene (=δ-guaiene |
| 12.451% | α-terpineol |
| 11.958% | trans-nerolidol |
| 10.763% | β-bisabolene |
| 9.944% | trans-pinene hydrate |
| 7.095% | valencene |
| 6.574% | caryophyllene oxide |
| 5.416% | β-sesquiphellandrene |
| 5.398% | borneol |
| 3.819% | cis-α-bergamotene |
| 3.156% | α-guaiene |
| 2.966% | 5-epi-7-epi-α-eudesmol |
| 2.830% | camphene |
| 2.487% | trans-β-ocimene |
| 1.710% | heptanal |
| 1.547% | fenchone |

Cannabis strain Midnight (60% saliva, 40% indica)
Terpenes:

| | |
|---|---|
| 100.000% | β-caryophyllene |
| 76.971% | α-eudesmol |
| 64.087% | guaiol |
| 61.661% | α-bisabolol |
| 61.065% | 10-epi-γ-eudesmol |
| 58.094% | bulnesol |
| 32.590% | α-humulene |
| 30.738% | trans-β-farnesene |
| 30.502% | myrcene |
| 21.718% | α-trans-bergamotene |
| 21.234% | linalool |
| 17.108% | limonene |
| 15.268% | β-bisabolene |
| 10.503% | α-terpineol |
| 9.310% | exo-fenchol |
| 7.191% | trans-pinene hydrate |
| 6.924% | β-sesquiphellandrene |
| 6.490% | trans, trans-α-farnesene |
| 6.254% | β-pinene |
| 5.447% | caryophyllene oxide |
| 5.001% | trans-nerolidol |
| 4.806% | 5-epi-7-epi-α-eudesmol |
| 3.773% | α-pinene |
| 3.402% | α-cis-bergamotene |
| 2.985% | borneol |
| 2.794% | valencene |
| 2.267% | selina-3,7(11)-diene |
| 1.676% | humulene epoxide II |
| 1.608% | γ-curcumene |
| 0.920% | fenchone |
| 0.857% | ipsdienol |
| 0.691% | camphene |
| 0.595% | heptanal |
| 0.541% | cis-β-farnesene |

Cannabis strain Dorit (70% indica, 30% sativa
Terpenes:

| | |
|---|---|
| 100.000% | β-caryophyllene |
| 67.723% | selina-4,7(11)-diene |
| 45.601% | germacrene B |
| 45.346% | β-cudesmol |
| 41.805% | 10-epi-γ-eudesmol |
| 41.611% | α-humulene |
| 36.077% | γ-elemene |
| 34.231% | epi-α-bisabolol |
| 34.132% | guaiol |
| 27.895% | bulnesol |
| 25.963% | trans-β-farnesene |
| 22.877% | α-pinene |
| 17.938% | myrcene |
| 17.501% | trans-α-bergamotene |
| 9.508% | β-pinene |
| 8.919% | β-bisabolene |
| 7.801% | caryophyllene oxide |
| 7.404% | juniper camphor |
| 6.901% | β-selinene |
| 6.337% | α-selinene |
| 4.423% | limonene |
| 3.750% | trans, trans-α-famesene |
| 3.670% | cis-α-bergamotene |
| 3.363% | linalool |
| 3.229% | β-sesquiphellandrene |
| 3.128% | humulene epoxide II |
| 2.607% | 5-epi-7-epi-α-eudesmol |
| 2.439% | γ-curcumene |
| 1.926% | endo-fenehol |
| 1.862% | α-terpineol |
| 1.772% | heptanal |
| 1.312% | trans-pinene hydrate |
| 0.995% | α-ylangene |
| 0.810% | camphene |
| 0.930% | trans-β-ocimene |
| 0.802% | borneol |
| 0.470% | β-phellandrene |
| 0.456% | 1,8-cineole |

1-48. (canceled)

49. A method for treating, alleviating or reducing at least one symptom of Inflammatory Bowel Disease (IBD) in a patient in need thereof, said method comprising administering to the patient at least one composition comprising:
a cannabis plant material comprising between about 16-24% (w/w) Tetrahydrocannabinol (THC), up to about 3% (w/w) Cannabidiol (CBD) and optionally up to about 1% (w/w) Cannabinol (CBN).

50. The method of claim 49, wherein said treating, alleviating or reducing at least one symptom of IBD comprises measuring in said patient a reduction of at least one of
(a) a score according to Disease Activity Index (DAI),
(b) Simple Endoscopic Score for Crohn's Disease (SES-CD),
(c) a level of an inflammatory marker in blood and/or a fecal sample,
(d) an improvement of at least one of weight, self-reporting on pain, bowel movement, quality of life.

51. The method of claim 49 wherein the patient is suffering from Crohn's disease.

52. The method of claim 49 wherein the patient is suffering from colitis.

53. The method of claim 49, wherein said method further comprises administering at least one drug for the treatment of IBD, simultaneously or in succession.

54. The method of claim 49, wherein said at least one composition comprises a cannabis plant material in the form of a dry plant material or an oil extract thereof.

55. The method of claim 54 wherein said at least composition is administered orally, by smoking, inhalation, vaporization or a combination thereof.

56. The method of claim 49, wherein said treating, alleviating or reducing of at least one symptom of IBD is immediate.

57. The method of claim 54, wherein said at least one composition comprises a cannabis plant material derived from a cannabis strain herein designated Erez, Alaska, Eran-Almog, Dorit, Omer, Shira, Or, Zohar, Barak, Tal, Jasmine, or a combination thereof.

58. The method of claim 54, wherein the composition comprises a cannabis plant material derived from a cannabis strain herein designated Erez, and the composition is administered by smoking, inhalation, vaporization or a combination thereof.

59. The method of claim 58, wherein the composition is administered to the patient suffering from colitis.

60. The method of claim 49 further comprising administering to the patient
(i) at least composition comprising a cannabis plant material comprising THC and CBD in amounts that are substantially equal or with THC:CBD ratio of about 1:1, and/or
(ii) at least one composition comprising a cannabis plant material enriched in CBD, the composition in (i) and the composition in (ii) are administered alone or in combination.

61. The method of claim 60, wherein said at least one composition in (i) comprises a cannabis plant material comprising between about 6-14% (w/w) THC and between about 6-16% (w/w) CBD, and said at least one composition in (ii) comprises a cannabis plant material comprising between about 14-24% (w/w) CBD and up to about 4% THC (w/w).

62. The method of claim 61, wherein said at least one composition in (i) and the at least one composition in (ii) further comprise up to about 1% CBN (w/w).

63. The method of claim 62, wherein said at least one composition in (i) comprises a cannabis plant material derived from a cannabis strain herein designated Midnight, Elna or Mango or a combination thereof, and said at least one composition in (ii) comprises a cannabis plant material derived from a cannabis strain herein designated Avidekel or Rephael or a combination thereof.

64. The method of claim 63, wherein said at least one composition in (i) is administered by smoking, inhalation, vaporization or a combination thereof, and said at least one composition in (ii) is administered via an oral route.

65. The method of claim 58 further comprising administering to the patient a composition comprising a plant material derived from the strain Midnight or a composition comprising a plant material derived from the strain Avidekel, administered alone or in combination, in succession to the composition with the strain Erez.

66. The method of claim 49 wherein said at least one composition further comprises at least one monoterpene selected from myrcene, limonene and pinene and at least one sesquiterpene selected from caryophyllene, guaiol and farnesene.
